(19) United States
(12) Patent Application Publication
(10) Pub. No.: US 2010/0291550 A1
(43) Pub. Date: Nov. 18, 2010

(54) Methods and compositions for modulating hair growth

(75) Inventor: Evgeny I. Rogaev, Shrewsbury, MA (US)

Correspondence Address: WOLF GREENFIELD & SACKS, P.C., 600 ATLANTIC AVENUE, BOSTON, MA, 02210-2206, US

(73) Assignee: University of Massachusetts, Boston, MA (US)

(21) Appl. No.: 12/311,681

(22) PCT Filed: Oct. 11, 2007
(86) PCT No.: PCT/US07/21747

§ 371 Date: Jan. 28, 2010

Related U.S. Application Data
(60) Provisional application No. 60/850,957, filed on Oct. 11, 2006.
(60) Provisional application No. 60/852,605, filed on Oct. 18, 2006.

(51) Int. Cl. C12Q 1/68; G01N 33/573; C07H 21/04; C12N 15/63; C12N 5/10; C12N 9/20

(52) U.S. Cl. 435/6; 435/7.4; 536/23.2; 435/320.1; 435/325; 435/198; 435/7.6

(57) ABSTRACT

The invention relates, in part, to methods and compounds that are useful to modulate hair growth in mammals. The invention, in part, also includes nucleic acids, polypeptides, and genetic constructs that may be used to diagnose and treat hair growth disorders, for screening for compounds that modulate hair growth, and for selecting treatment regimens for subjects with hair loss disorders or hair growth conditions. The invention in some aspects also includes kits that may include polypeptides and/or nucleic acids for diagnosis of hair growth disorders and/or for the modulation of hair growth in subjects.

### RELATED APPLICATIONS

This application claims the benefit under §119(e) of U.S. provisional application 60/850,957, filed Oct. 11, 2006, and U.S. provisional app 60/852,605, filed Oct. 18, 2006 the entire disclosures of which are incorporated herein by reference.

### FIELD OF THE INVENTION

The invention relates, in part, to methods and compounds that are useful to modulate hair growth in mammals. The invention, in part, includes polypeptides, nucleic acids and genetic constructs that may be used to diagnose hair growth disorders, to modulate hair growth, and to screen for compounds that modulate hair growth. The invention in some aspects also includes kits that may include polypeptides and/or nucleic acids for diagnosis of hair growth disorders and/or for the modulation of hair growth in subjects. Polypeptide and/or nucleic acid molecules of the invention may also be useful for research purposes such as for identifying compounds that modulate hair growth.

### BACKGROUND OF THE INVENTION

Mammalian hair follicles are self-renewing organs spread over the body. These structures are interesting models of regulation and differentiation of stem cells. The hair follicle cycle includes anagen (the duration of hair growth), catagen (stage of involution) and telogen (period of rest) stages. The hair follicles contain multipotent keratinocyte stem cells located in a niche of the hair termed “bulge” and are thus capable of reconstituting themselves (1,2). A unique feature of these stem cells is that they return to the quiescent state after giving rise to the progenitor matrix cells required for the production of new hairs. The body distribution, size and number of hair follicles in humans is distinct from that of other mammalian species (3,4). It is not yet completely understood which molecules control recycling, differentiation and morphogenesis of human hair follicles and what causes progressive hair loss on the scalp over a lifetime.

The age, gender, and other genetic and non-genetic (e.g., stress) factors contribute to hair loss, which is most prominent in males but occurs also in females. Two mutant human genes for rare familial cases of alopecia and hypotrichosis were identified with help from mouse genetic studies. The mutations in the human gene homologous to the murine hairless gene underlie alopecia universalis (4). Defects in desmoglin 4 gene were found in some pedigrees with alopecia and in mouse strain with lanceolate phenotype (5,6). Nonsense mutations were found in the gene coraeodesmosin (CDSN) in families for hypotrichosis simplex with loss of scalp hairs but normal body hairs (7).

### SUMMARY OF THE INVENTION

The invention provides methods and compositions to diagnose hair loss disorders and to modulate hair growth in a subject. The invention is based on the surprising identification of the LIPH gene and its encoded polypeptide in a pathway associated with hair growth in hair follicle cells. It has now been discovered that alterations in the LIPH genes and polypeptides in the pathway may result in disorders of hair growth. The identified genes and polypeptides can be utilized in methods and compositions that modulate hair growth in a subject. In some aspects of the invention, the identification of the genes permits diagnosis of hair loss disorders and the selection of treatment for subject with hair growth disorders or conditions. The invention in some aspects includes kits that include polypeptides and/or nucleic acids of the invention that are involved in the hair growth pathway that comprises LIPH genes and polypeptides. In some aspects of the invention, the polypeptide and/or nucleic acid molecules of the invention are useful to increase hair growth in a subject who has or is suspected of having a hair growth disorder and/or in a subject who does not have and is not suspected of having a hair growth disorder.

The invention, in part, relates to the identification of a gene responsible for regulation of hair growth in humans; the identification of a novel biochemical pathway controlling hair growth and re-cycling; prediction of small molecules that may regulate scalp hair and body hair growth and that may be compounds for therapy of common and rare genetic forms of hair-loss (baldness); and the prediction of molecules that may enhance or inhibit hair growth in human and mammalian animals.

According to one aspect of the invention, methods for diagnosing an LIPH-associated hair growth disorder in subject are provided. The methods include assessing an LIPH gene or LIPH gene expression in a sample from the subject, wherein the presence of an alteration in the LIPH gene such that the gene encodes or expresses an LIPH polypeptide having a modified activity as compared to a wild-type LIPH polypeptide is diagnostic for an LIPH-associated hair growth disorder in the subject. In some embodiments, the LIPH gene is assessed by determining a nucleic acid sequence of the gene. In certain embodiments, the LIPH gene expression is assessed by determining an amino acid sequence of a polypeptide encoded by the LIPH gene. In some embodiments, the LIPH gene expression is assessed by determining the level of LIPH mRNA or LIPH polypeptide in the cell. In some embodiments, the alteration is in an exon of the LIPH gene sequence. In certain embodiments, the exon is exon 4 of the LIPH gene. In some embodiments, the alteration in the LIPH gene sequence includes a deletion, addition, or mutation of one or more nucleotides. In some embodiments, the alternation of exon 4 is a deletion that includes all of the nucleotides of exon 4 of the LIPH gene. In certain embodiments, the alteration of exon 4 includes the deletion or mutation of nucleotides that encode one or more of the Ser-154, Asp-178, and/or His-248 residues of the LIPH polypeptide. In some embodiments, the alteration of exon 4 includes the deletion or mutation of nucleotides that encode the Ser-154, Asp-178, to and/or His-248 residues of the LIPH polypeptide. In some embodiments, the subject is human. In certain embodiments, the LIPH-associated hair growth disorder is hypotrichosis. In some embodiments, the LIPH-associated hair growth disorder is alopecia. In some embodiments, the method also includes determining a treatment for the subject based, in part, on the diagnosis of the LIPH-associated hair growth disorder. In certain embodiments, the treatment includes modifying a pathway that includes the LIPH polypeptide in a hair follicle cell of the subject an amount effective to modulate hair growth in the subject. In some embodiments, modifying the pathway that includes the LIPH polypeptide includes increasing a level of a simple lipid in the hair follicle cell. In some embodiments, the simple lipid is a lysophosphatidic acid (LPA). In some embodiments, the LPA is 1- or 2-acyl-lysophosphatidic acid. In certain embodiments, the simple lipid is a modified LPA. In some embodiments, the level of the simple lipid is increased in the hair follicle cell by administering the simple lipid to the subject. In some embodiments, the level of the simple lipid is increased in the hair follicle cell by inducing expression of an endogenous and/or exogenous LIPH polypeptide in the hair follicle cell. In certain embodiments, the LIPH polypeptide is a polypeptide encoded by a wild-type LIPH gene, an isoform of a wild-type LIPH gene, a mutant LIPH gene, or a gene allelic to an LIPH gene. In some embodiments, modifying the pathway that includes the LIPH polypeptide includes increasing an LIPH polypeptide in a hair follicle cell of the subject. In some embodiments, increasing the LIPH polypeptide includes inducing expression of exogenous or endogenous LIPH polypeptide in the hair follicle cell. In certain embodiments, modifying the pathway includes LIPH polypeptide includes increasing activity or expression of a homolog of LIPH. In some embodiments, modifying the pathway that includes LIPH polypeptide includes increasing levels of a molecule that generates PA. In some embodiments, modifying the pathway that includes an LIPH polypeptide includes increasing activity or expression of a molecule that is a phospholipase precursor.

According to another aspect of the invention, kits for diagnosing a LIPH-associated hair growth disorder in a subject are provided. The kits include a container containing a compound to assess an LIPH gene or an LIPH polypeptide in a hair follicle from the subject. In some embodiments, the LIPH gene is assessed by determining a nucleic acid sequence of the gene. In certain embodiments, the LIPH gene is assessed by determining an amino acid sequence of a polypeptide encoded by the LIPH gene. In some embodiments, the LIPH gene is assessed by determining the level of LIPH mRNA or LIPH polypeptide in the cell. In some embodiments, the LIPH gene is assessed for an alteration in the sequence of the LIPH gene sequence. In certain embodiments, the alteration is in an exon of the LIPH gene. In some embodiments, the exon is exon 4 of the LIPH gene. In some embodiments, the alteration in the sequence of the LIPH gene includes a deletion, addition, or mutation of one or more nucleotides. In certain embodiments, the alternation of exon 4 includes a deletion of all of the nucleotides of exon 4 of the LIPH gene. In some embodiments, the alteration of exon 4 includes the deletion or mutation of nucleotides that encode one or more of the Ser-154, Asp-178, and/or His-248 residues of the LIPH polypeptide. In some embodiments, the alteration of exon 4 includes the deletion or mutation of nucleotides that encode the Ser-154, Asp-178, and/or His-248 residues of the LIPH polypeptide. In certain embodiments, the subject is human. In some embodiments, the LIPH-associated hair growth disorder is hypotrichosis. In some embodiments, the LIPH-associated hair growth disorder is alopecia.

According to yet another aspect of the invention, isolated nucleic acid molecules that encode (a) an LIPH polypeptide that possesses a lipase activity that is reduced from the level of lipase activity of a wild-type LIPH polypeptide, or (b) a full-length complement of thereof are provided. In some embodiments, the LIPH gene encodes an LIPH polypeptide having a deletion or substitution of all or part of the LIPH polypeptide sequence encoded by exon 4 of the LIPH gene. In certain embodiments, the deletion or substitution includes a deletion or substitution of one or more of the Ser-154, Asp-178, and/or His-248 residues of the encoded LIPH polypeptide. In some embodiments, the deletion or substitution includes a deletion or substitution of the Ser-154, Asp-178, and/or His-248 residues of the encoded LIPH polypeptide. In some aspects of the invention, any of the aforementioned isolated nucleic acid molecules are operably linked to a promoter. In certain aspects of the invention a host cell transformed or transfected with any of the aforementioned expression vectors is provided. In some embodiments, the host cell is a hair follicle cell. According to another aspect of the invention, an isolated polypeptide encoded by any of the aforementioned isolated nucleic acid molecules is provided. In some embodiments, the isolated polypeptide includes an amino acid sequence encoded by the LIPH gene that is missing exon 4.

According to yet another aspect of the invention, methods for identifying a compound for increasing hair growth are provide. The methods include contacting the compound with a cell that includes a mutated LIPH gene, wherein the mutation reduces activity of LIPH polypeptide in the cell compared to a control cell with a non-mutated LIPH gene, and determining the effect of the compound on LIPH polypeptide activity of the cell, wherein a compound that increases LIPH polypeptide activity of the cell is identified as a compound that increases hair growth. In some embodiments, the mutant LIPH gene encodes an LIPH polypeptide having a deletion or substitution of all or part of the LIPH polypeptide sequence. In certain embodiments, the deletion or substitution is in an exon of the LPH gene. In some embodiments, the exon is exon 4 of the LIPH gene. In some embodiments, the deletion includes all of exon 4 of the LIPH gene. In some embodiments, the deletion or substitution includes a deletion or substitution of one or more of the Ser-154, Asp-178, and/or His-248 residues of the encoded LIPH polypeptide. In certain embodiments, the deletion or substitution includes a deletion or substitution of the Ser-154, Asp-178, and/or His-248 residues of the encoded LIPH polypeptide. In some embodiments, the cell is a hair follicle cell.

### BRIEF DESCRIPTION OF THE DRAWINGS

FIG. 1 is a schematic diagram showing the procedure for positional cloning of gene for hypotrichosis. FIG. 1A shows the STR genotyping identified a linkage to 3q27. The common haplotypes persisting in affected individuals and critical recombinations refining the linkage interval are shown. FIG. 1B shows the genetic map and genes assigned in mapped region. The markers in bold are new markers isolated in this study. The recombination events define (CA)₅ as centromeric and D3S1350 as telomeric boundaries of the minimal genetic interval. The grey and black bar denotes the region of shared haplotypes for Mari mutant carriers and black bar is the region of shared haplotypes between all affected individuals in two populations. The four genes in this region are located. The gene sequencing analysis of LIPH(PCR primers are shown by arrows) revealed the deletion of exon 4 and flanked intronic sequences. The correspondence of putative catalytic amino acid-residues to LIPH exons is shown. The introns 3 and 5 are enriched by members of families of SINE retroposons. The uneven recombination event between ALU-repeat copies leading to the deletion is shown.

FIG. 2 provides a graph showing real-time PCR analysis with normalization to other housekeeping gene confirmed the prominent expression of LIPH in hair follicles. S. skin—adult scalp skin, matrix—matrix area of plucked follicles, an.follicle—anagen whole plucked follicle, an.bulge—dissected bulge region of anagen follicles which contains stem cells, bulb—bulb region of plucked follicles which contains matrix cells, telogen—plucked telogen follicles which contains stem cell region, DP—dermal papilla cells after 0, 1 or 12 days of to culturing; the samples marked as PO are primary cultured keratinocytes from corresponding follicle areas.

FIG. 3 shows a comparison of sequences of a family of triglyceride lipases in Homo Sapiens. The figure demonstrates conservation and the putative functional pathway of LIPH. FIG. 3A shows that the alignment of the exon 4 region of LIPH with other members of the triglyceride lipase family demonstrated that exon 4 encodes a highly conserved domain containing evolutionary invariant amino acid residues. The aspartic acid at position 178 along with serine-154 and histidine-248 are active catalytic residues conserved in all lipases. LIPH is SEQ ID NO:17; LIPI is SEQ ID NO:18; PS-PLA1 is SEQ ID NO:19; LIPG is SEQ ID NO:20; LPL is SEQ ID NO:21; LIPC is SEQ ID NO:22; PNLIP is SEQ ID NO:23; PNLIPRP1 is SEQ ID NO:24; and PNLIPRP2 is SEQ ID NO:25. FIG. 3B shows the predicted catalytic role of LIP in synthesis of bioactive LPA supposed to be abolished in affected individuals.

FIG. 4 provides tables showing parametric LOD Scores. FIG. 4A shows two-point LOD scores for the linkage of hypotrichosis to STR markers on chromosome 3g27. FIG. 4B shows multipoint LOD scores in homozygous interval and in flanking genomic regions calculated for Mari families. High Multiple Lod Scores were obtained when summarized data for all Mari and Chuvash families in the homozygosity CA4-D3S1530 interval (Multiple Lod Scores: 17.04018.027).

FIG. 5 shows representative diagrams of strategy for positional cloning of the mutant LIPH gene. FIG. 5A shows representative haplotypes for the STR markers showing linkage to hypotrichosis mutation in families from Mari and Chuvash populations. The Mari and Chuvash affected individuals from unrelated families share the common haplotypes associated with the disorder. Two rare recombination events in Mari and Chuvash individuals defined the minimal genomic interval for the mutant gene between D3 S 1530 and CA5 markers. FIG. 5B shows genetic and physical map of the chromosome 3g27 locus showing linkage to the hypotrichosis. The black bars are the centromeric to telomeric intervals defined by critical recombination events in Mari and Chuvash populations. STRs in black indicate markers isolated in this study.

FIG. 6 shows multiple sequencing alignment of the human LIPH with orthologous members of LIPH family from the following vertebrate species: H.sap.—Homo sapiens NP_{—}640341 (SEQ ID NO:94); P.trog.—Pan troglodytes XP_{—}516924 (SEQ ID NO:95); B.taur.—Bos taurus XP_{—}589466 (SEQ ID NO:96); C.fam.—Canis familiaris XP_{—}545236 (SEQ ID NO:97); M.mus.—Mus musculus AAM18804 (SEQ ID NO:98); X.trop. Xenopus tropicalis NP_{—}001011098 (SEQ ID NO:99); D.rer.—Danio rerio XP_{—}687645 (SEQ ID NO:100).

FIG. 7 shows sequencing analysis of genomic region containing exon 4 and flanking introns of LIPH gene.

### PARTIAL LISTING OF SEQUENCES

SEQ ID NO:1 is LIPH (homo sapiens) Genbank Accession No. AAH64941: 451 amino acids
SEQ ID NO:2 is LIPH wild-type genomic sequence; 1-2114 bp; intron 3-4 1-1358 bp; exon 4 1358-1460 bp; intron 4-5 1461-2114 bp
SEQ ID NO:3: is patient (homo sapien) C keratinocytes—LIPH protein—418 amino acids
SEQ ID NO:4 is LIPH Deletion mutant genomic—exon 4 deletion 1-983 bp; recombination region 782-814 bp; intron 3-4 1-814 bp; intron 4-5 815-983 bp.
SEQ ID NO:5 is LIPH sequence wild-type Ex 1; 1-99; exon 1-56; coding exon 8-56; intron 1-2 57-99.
SEQ ID NO:6 is LIPH sequence wild-type Ex 2; 1-432; intron 1-2 1-30; exon 2 31-398; intron 2-3 399-432.
SEQ ID NO:7 is LIPH sequence Ex 3; 1-201; intron 2-3 1-29; exon 3 30-138; intron 3-4 139-201.
SEQ ID NO:8 is LIPH sequence Ex 5; 1-154; intron 4-5 1-30; exon 5 31-120 intron 5-6 121-154.
SEQ ID NO:9 is LIPH sequence Ex 6 1-256; intron 5-6 1-77; exon 6 78-245; intron 6-7 246-256.
SEQ ID NO:10 is LIPH sequence Ex 7 1-226; intron 6-7 1-24; exon 7 25-120; intron 7-8 121-226.
SEQ ID NO:11 is LIPH sequence Ex 8 1-182; intron 7-8 1-53; exon 8 54-165; intron 8-9 166-182.
SEQ ID NO:12 is LIPH sequence Ex 9 1-283; intron 8-9 1-92; exon 9 93-266; intron 9-10 267-283.
SEQ ID NO:13 is LIPH sequence Ex 10 1-224; intron 9-10 1-90; exon 10 91-224
SEQ ID NO:14 is fragment of SEQ ID NO:2
SEQ ID NO:15 is fragment of SEQ ID NO:2
SEQ ID NO: 16 is fragment of SEQ ID NO:4
SEQ ID NO: 17 is fragment of LIPH, fragment of SEQ ID NO:1
SEQ ID NO: 18 is fragment of LIPI
SEQ ID NO: 19 is fragment of PS-PLA1
SEQ ID NO: 20 is fragment of LIPG
SEQ ID NO: 21 is fragment of LPL
SEQ ID NO: 22 is fragment of LIPC
SEQ ID NO: 23 is fragment of PNLIP
SEQ ID NO: 24 is fragment of PNLIPRP1
SEQ ID NO: 25 is fragment of PNLIPRP2
SEQ ID NO:26 is wild-type patient—keratinocytes; 2065 bp; CDS—88-1254 bp; 5′ UTR—1-87; 3′utr′ 1255-2065
SEQ ID NO:27 is cDNA—pancreas human; 1506 bp; CDS—88-1443 bp; 5′ utr 1-87; 3′utr 1444-1506 bp

### DETAILED DESCRIPTION OF THE INVENTION

The invention described herein relates to methods and compositions to modulate (e.g., alter) hair growth in a subject. A polypeptide and its encoding nucleic acid molecule that are associated with the hair growth pathway in hair follicle cells have been identified. The identified polypeptides and/or their encoding nucleic acids can be utilized in methods and compositions that modulate hair growth in a subject and can be used to diagnose and/or treat a hair growth disorder. The invention also includes kits that include the polypeptides and/or nucleic acids of the invention that modulate hair growth and can be used to diagnose and/or treat a hair growth disorder. In some aspects of the invention, the polypeptide and/or nucleic acid molecules of the invention are useful to increase hair growth in a subject who has or is suspected of having a hair growth disorder. In certain embodiments of the invention, polypeptides and/or nucleic acid molecules of the invention are useful to increase hair growth in a subject who does not have and is not suspected of having a hair growth disorder.

The LIPH gene has never been implicated before as a factor regulating hair follicles or hair growth regulation. The common function of the gene and related homologous genes (phospholipases, members of triglyceride lipase family) is hydrolysis of lipids, particularly phospholipids. This pathway has never been suggested before in control of hair follicle and hair growth. The loss of function of this gene (phospholipase) or related homologs have not been suggested before as a cause of hair growth deficiency and hair loss. The identification of the LIPH gene and polypeptide in a hair growth pathway in hair follicle cells is novel and permits prediction of novel molecules, such as lipid products of lipase hydrolysis for treatment of hair diseases and regulation of hair growth in subjects with normal hair growth (e.g., subjects without a hair growth disorder). The identification of the LIPH gene and polypeptide in a hair growth pathway also permit assessment of the LIPH genes and mutations of the gene for diagnosis of hair loss and for determining treatment strategy for hair growth disorders or conditions.

As used herein the terms “treat” and “treatment” mean prophylactic treatment as well as treatment of a present disorder or condition. As used herein, a subject without a hair growth disorder, to whom a treatment to modulate hair growth may be applied, may be considered to have a “hair growth condition”, which need not be a disease or disorder. Thus, a subject for whom addition hair growth is desired, even though that subject does not have a hair growth disorder, may be referred to as having a hair growth condition.

The invention is based in part, on the identification of a small chromosomal region linked to hair growth phenotype; a gene controlling hair growth; and mutant isoforms of this gene underlying defects in hair growth. A strategy of positional cloning was utilized for epidemiological and family screening in human populations. Fine mapping was performed and mutation screen for the LIPH gene alleles was performed. The invention, in part, includes methods for genetic modifications of the Lipase H (LIPH) gene in animals including disruption of the gene; transgenic expression of the wild-type and mutant or spliced isoforms of the LIPH genes for purposes of modification of hair and fur growth in animals, including, but not limited to humans. In addition, the invention relates to methods and kits for diagnostic application of the allelic variants of LIPH genes to predict hair phenotypes and hair-loss-disorders, and for the determination of treatment for hair loss disorders and/or conditions. The invention also relates in part to methods to identify additional genes and proteins that interact with LIPH in hair growth pathways. For example the use of functional assays or genetic assays and the assessment of polypeptides that physically interact with LIPH and may therefore contribute to hair growth regulation. In some aspects the invention may also include methods for application of simple lipids, including but not limited to lysophoshatidic acids, phospholipids, iysophosphatidic acids and their natural and synthetic modifications, for regulation of hair growth and treatment of different forms of baldness. The invention also relates in part to the identification of allelic forms of LIPH gene that partially or completely disrupts function of an LIPH gene underlying a deficiency of hair growth. The invention also relates in part, to genetic constructs of LIPH, with and without mutations, wherein the genetic construct may be expressed in animals to modify their hair phenotype. In some aspects the invention includes methods to identify compounds to up-regulate LIPH for purposes to increase hair growth and development.

Additional aspect of the invention relate, in part, to the development and/or use of molecules, techniques, solutions, and/or creams to prevent or treat scalp hair loss and to enhance deficient hair growth in rare genetic disorders, in common forms of baldness, in chemotherapy related hair-loss, in hair-loss related to ageing processes in humans and non-human mammals. Additionally, methods and compositions of the invention may be used to develop and produce domestic, agriculture and laboratory animal strains and lineages with modified fur or hair growth (e.g., increased hair growth). The invention also includes, in some aspects, diagnostic tests and molecular kits for determining individual or group risk factors for hair loss. The invention also relates to targeting specific molecules (LIPH and LPA pathways) to modify certain pathways, stem cell differentiation and hair follicle cell transformation and proliferation.

The invention described herein relates to the identification and use of polypeptides that are involved in a pathway associated with hair growth. It has now been identified that LIPH polypeptides are involved in a pathway associated with hair growth and this identification allows use of these newly identified hair-growth associated polypeptides and/or their encoding nucleic acid molecules in diagnostic assays for hair growth, compositions for treating and assessing hair growth disorders, methods for treating of hair growth disorders, methods for screening for compounds to treat hair growth disorders, and in diagnostic and treatment kits and assays. Such assays and kits are useful to diagnose hair growth disorders in human and animal subjects. The methods and kits described herein may also be used to evaluate treatments for hair growth disorders.

It will be understood that the invention also encompasses the use of methods and compositions other than LIPH that may be used to modulate the pathway that comprises LIPH and is associated with hair growth. Thus, the invention includes, in part, methods and compositions to alter the pathway upstream and/or downstream of LIPH activity in a manner to modulate hair growth. It will be understood that the strategies and methods described throughout herein for altering, modifying, delivering, and using LIPH polypeptides and their encoding nucleic acids to modulate hair growth may also be applied to other polypeptides that function in the pathway that comprises LIPH and is associated with hair growth.

An LIPH polypeptide is a hair-growth-associated polypeptide, and is involved in a hair growth pathway in hair follicles. The amino acid sequence of wild-type, human LIPH polypeptide is set forth herein as SEQ ID NO:1. The invention also relates to the use of nucleic acid molecules that encode LIPH polypeptides in compositions and methods. A genomic DNA sequence that encodes SEQ ID NO:1 is provided as SEQ ID NO:2. A newly identified LIPH polypeptide that is associated with hair growth disorders lacks the amino acid sequence that is encoded by exon 4 of the LIPH gene is set forth herein as SEQ ID NO:3 and the genomic DNA that encodes SEQ ID NO:3 is set forth herein as SEQ ID NO:4. It will be understood that as used herein the term “LIPH polypeptide” may refer to a full-length or fragment of a wild-type or mutant LIPH polypeptide and the term “LIPH nucleic acid” may refer to a full-length or fragment of a wild-type or mutant LIPH nucleic acid.

The present invention, in one aspect, involves cloning of cDNAs that encode a LIPH polypeptide that is involved in a hair growth pathway of a hair follicle cell. The invention involves in one aspect, human and other mammalian polypeptides, nucleic acid molecules encoding those polypeptides, functional modifications and variants of the foregoing, useful fragments of the foregoing, as well as therapeutic and diagnostic products (including antibodies), and methods relating thereto.

Lipase H (LIPH) is expressed in human and animal cells. The lipase H polypeptide (LIPH) is also known in the art as membrane-associated phosphatidic acid-selective phospholipase A1 (MPAPLA1), phospholipase A1, and phosphatidic acid-selective membrane-associated LPD lipase related protein, (LPDLR). The LIPH gene has been mapped to chromosome 3Q27-q28 and contains 10 exons and spans about 45 kb (Jin, W.; et al., Genomics 80: 268-273, 2002). LIPH is a phosphatidic acid-selective phospholipase A1 (PLA1) that produces 2-acyl lysophosphatidic acid (LPA).

It has now been discovered that a mutation in the LIPH gene that results in a deletion of exon 4 of the sequence, results in an LIPH polypeptide that lacks LIPH lipase function. This mutated gene has now been found to be expressed in subjects with hair growth disorder, and the loss of LIPH function is correlated with the presence of hair growth abnormalities in mammals.

Mammalian hair follicles are self-renewing organs spread over the body. The hair follicle cycle includes anagen (the duration of hair growth), catagen (stage of involution) and telogen (period of rest) stages. The hair follicles contain multipotent keratinocyte stem cells located in a niche of the hair termed “bulge” and are thus capable of reconstituting themselves (1,2). Hair cells are able to return to the quiescent state after giving rise to the progenitor matrix cells required for the production of new hairs.

Different aspects of hair growth that can be assessed, include, for example the growth of a hair and the maintenance of a hair. As used herein, “growth” of a hair means the process of growing a hair and the term “maintenance” of a hair means keeping the hair. For example, some hair growth disorders may be characterized by a lack of hair growth and some hair growth disorders may be characterized by the loss of a hair that has grown or is growing. It will be understood that some hair growth disorders may be characterized by a deficit in hair growth and/or by an increase in loss of hair that is growing or has grown.

A hair growth disorder may be a disorder in which a reduced amount of hair is grown and/or maintained by a subject (versus a normal control). Examples of hair growth disorders that include a reduction in the amount of hair grown and/or maintained, although not intended to be limiting, are hypotrichosis, alopecia, alopecia areata, androgenetic alopecia, anagen effluvium, self-induced hair loss, telogen effluvium, scarring alopecia, aging, and drug- or chemical-induced hair loss (e.g., resulting from chemotherapy agents, etc.). In some embodiments of the invention a subject has been diagnosed with a hair growth disorder.

As used herein, a subject may be a mammal, and may be a human, non-human primate, cow, horse, pig, sheep, goat, dog, cat, or rodent, including but not limited to: guinea pig, rat, and mouse. In some embodiments of the invention, a subject has a hair growth disorder, and in some embodiments a subject does not have a hair growth disorder.

It will also be understood that method, compositions, and/or products of the invention may be used to modulate hair growth in a subject without a diagnosed or suspected hair growth disorder. It may be desirable to increase an amount of hair grown on some or all regions of a subject's body. As used herein, the term “abnormal” refers to decreased hair growth and/or hair maintenance (including zero growth or maintenance) as compared to hair growth in a subject who does not have a hair growth-associated disorder. Abnormal hair growth may be correlated to abnormalities of a pathway that includes the LIPH polypeptide in hair follicle cells. An example of an abnormality in such a pathway is the expression of a mutant form of LIPH, wherein the presence of the mutated form of LIPH polypeptide results in a change in hair growth and/or hair maintenance. For example, expression of an LIPH polypeptide that lacks all or part of the sequence encoded by exon 4 of the LIPH gene, results in abnormally low hair production and maintenance. Abnormal expression of an LIPH polypeptide may refer to expression of a mutant LIPH polypeptide or an abnormal amount of expression of a wild-type LIPH polypeptide as compared to its expression in a subject who does not have a hair growth disorder. For example, abnormal expression may be expression of wild-type LIPH that is not about 100% of the level of wild-type LIPH in a subject free of a hair growth-associated disorder. In another example, the level of wild-type LIPH expression could be outside of the range of expected levels in normal subjects. In another example, abnormal expression may be expression of a mutant LIPH polypeptide zero LIPH polypeptide or any amount statistically significantly less than the expression of a normal level of wild-type LIPH polypeptide, e.g., in a normal subject.

Abnormal expression may be determined by comparing levels of LIPH molecules (nucleic acids and/or polypeptides) to those levels in controls. Importantly, levels of expression or activity of LIPH in a cell or subject may be determined using the methods provided herein and may be advantageously compared to controls according to the invention. Controls may include positive and negative control that may be a predetermined value that can take a variety of forms. A control can be a single cut-off value, such as a median or mean. It can be established based upon comparative groups, such as in groups having normal amounts of LIPH expression and/or activity and groups having abnormal amounts of LIPH expression and/or activity. Another example of comparative groups may be groups having normal and groups without abnormal hair growth. Another comparative group may be a group with a family history of abnormal hair growth and a group without such a family history. A predetermined value can be arranged, for example, where a tested population is divided equally (or unequally) into groups, such as a low-risk group, a medium-risk group and a high-risk group or into quadrants or quintiles, for example for a hair loss disorder, the lowest quadrant or quintile being individuals with the lowest risk and highest amounts of normal hair growth and/or normal LIPH activity and/or expression and the highest quadrant or quintile being individuals with the highest risk and lowest amount of normal hair growth and/or normal LIPH activity and/or expression.

The predetermined value, of course, will depend upon the particular population selected. For example, an apparently hair healthy population will have a different ‘normal’ range than will a population that is known to have a condition related to abnormal hair growth. Accordingly, the predetermined value selected may take into account the category in which an individual or cell falls. Appropriate ranges and categories can be selected with no more than routine experimentation by those of ordinary skill in the art. As used herein, “abnormal” means not normal as compared to a control. By abnormally high it is meant high relative to a selected control. By abnormally low it is mean low relative to a selected control. Typically a control will be based on apparently healthy normal individuals in an appropriate age bracket or apparently healthy cells.

It will also be understood that controls according to the invention may be, in addition to predetermined values, samples of materials tested in parallel with the experimental materials. Examples include samples from control populations or control samples generated through manufacture to be tested in parallel with the experimental samples.

As used herein, a compound that “modulates hair growth” refers to a compound that alters the pathway comprising LIPH in a follicle cell so that hair growth is different because of the contact with or the presence of the compound than the pathway would be if not contacted with or in the presence of the compound. In particular, such compounds may include small molecules such as simple lipids, may include antibodies, etc. A compound of the invention that modulates hair growth may affect the pathway comprising LIPH in a follicle cell by affecting LIPH activity or may act upstream or downstream of the LIPH to alter hair growth. A compound of the invention that modulates hair growth may include a nucleic acid that encodes a LIPH polypeptide (or other pathway-associated polypeptide). A compound of the invention that modulates hair growth may be an LIPH polypeptide or other pathway-associated polypeptide. As used herein, the term polypeptide is meant to include large molecular weight proteins and polypeptides and low molecular weight polypeptides or fragments thereof. As used herein, an LIPH polypeptide may be a full-length LIPH polypeptide or a fragment thereof.

According to one aspect of the invention, an isolated LIPH nucleic acid molecule is provided. The isolated nucleic acid molecule may be a nucleic acid molecule or fragment thereof that is (a) a wild-type LIPH gene (e.g., SEQ ID NO:2) or a mutant LIPH gene (e.g., SEQ ID NO:4) and that codes for an LIPH polypeptide, (b) nucleic acid molecules that differ from the nucleic acid molecules of (a) in codon sequence due to the degeneracy of the genetic code, and (c) complements of (a), no more than about 18% of the nucleotides in (a) are changed from the nucleic acid sequence of the wild-type or mutant LIPH gene. Preferably, no more than about 17%, 16%, 15%, 14%, 13%, 12%, 11%, 10%, 9%, 8%, 7%, 6%, 5%, 4%, 3%, 2%, or 1% of the nucleotides are changed relative to the sequence of the wild-type LIPH gene or mutant gene in the nucleic acid molecules that hybridize under stringent conditions to the wild-type LIPH gene. The invention also includes mutant LIPH nucleic acids and polypeptides that possess reduced function or increased function in a hair growth pathway as compared to a wild-type LIPH nucleic acid or polypeptide.

In some embodiments of the invention an isolated LIPH nucleic acid of the invention is an LIPH nucleic acid molecule that encodes an LIPH polypeptide. As used herein, the term “LIPH polypeptide” may refer to a polypeptide or fragment thereof that is encoded by a wild-type LIPH gene or a fragment thereof or a mutant LIPH gene or a fragment thereof. A fragment of a wild-type or mutant LIPH nucleic acid may possess a functional activity of a wild-type LIPH nucleic acid. As used herein, an LIPH functional activity of a LIPH nucleic acid refers to participation of an LIPH nucleic acid in the pathway of hair production a hair follicle cell and may also include encoding a polypeptide that has a lipase activity of an LIPH polypeptide. LIPH functional activity can be determined, for example, as described in the Examples section herein, by monitoring hair growth in a cell and/or subject, and by other art-known methods of assessing polypeptide and/or nucleic acid levels and/or function.

Homologs and alleles of an LIPH gene are understood to be encompassed by the invention. The skilled artisan is familiar with the methodology for screening cells and libraries for expression of homolog and allelic molecules that then are routinely isolated, followed by isolation of the pertinent nucleic acid molecule and sequencing.

In general homologs and alleles typically will share at least 80% nucleotide identity and/or at least 80% amino acid identity to the wild-type LIPH gene sequence and the LIPH polypeptide sequence provided herein as SEQ ID NO: 1, and in some instances will share at least 81%, 82%, 83%, 84%, 85%, 86%, 87%, 88%, 89%, 90%, 91%, 92%, 93%, 94%, 95%, 96%, 97%, 98%, 99%, or 99.5% nucleotide identity and/or at least 81%, 82%, 83%, 84%, 85%, 86%, 87%, 88%, 89%, 90%, 91%, 92%, 93%, 94%, 95%, 96%, 97%, 98%, 99%, or 99.5% amino acid identity. The percent identity can be calculated using various publicly available software tools developed by NCBI (Bethesda, Md.) that can be obtained through the internet (ftp:/ncbi.nlm.nih.gov/pub/). Exemplary tools include the BLAST system available at http://www.ncbi.nlm.nih.gov, which uses algorithms developed by Altschul et al. (Nucleic Adds Res. 25:3389-3402, 1997). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acid molecules also are embraced by the invention.

In screening for LIPH genes, a Southern blot may be performed using the foregoing conditions, together with a detectably labeled probe (e.g. radioactive or chemiluminescent probes). After washing the membrane to which the DNA is finally transferred, the membrane can be placed against X-ray film or a phosphorimager to detect the radioactive or chemiluminescent signal. In screening for the expression of LIPH RNA, Northern blot hybridizations using the foregoing conditions can be performed on samples taken from subjects suspected of having a condition characterized by aberrant expression of a LIPH molecule, e.g., abnormal hair growth and/or abnormal LIPH polypeptide expression. Amplification protocols such as PCR using primers that hybridize to the sequences presented also can be used for detection of the LIPH genes or expression thereof.

Identification of related sequences can be achieved using PCR and other amplification techniques suitable for cloning related nucleic acid sequences. Preferably, PCR primers are selected to amplify portions of a nucleic acid sequence believed to be conserved (e.g., an evolutionarily conserved region in exon 4 of the wild-type LIPH gene, a portion of the LIPH gene that includes the putative catalytic amino acid triad of: Ser-154, Asp-178, and/or His-248), a ligand binding domain, etc.). Nucleic acids are preferably amplified from a tissue-specific library. One also can use expression cloning utilizing antisera to identify nucleic acids that encode related polypeptides.

The invention also includes degenerate nucleic acid molecules which include alternative codons to those present in the native materials. For example, serine residues are encoded by the codons TCA, AGT, TCC, TCG, TCT, and AGC. Each of the six codons is equivalent for the purposes of encoding a serine residue. Thus, it will be apparent to one of ordinary skill in the art that any of the serine-encoding nucleotide triplets may be employed to direct the polypeptide synthesis apparatus, in vitro or in vivo, to incorporate a serine residue into an elongating LIPH polypeptide. Similarly, nucleotide sequence triplets that encode other amino acid residues include, but are not limited to: CCA, CCC, CCG, and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

According to another aspect of the invention, further isolated nucleic acid molecules that are based on the above-noted LIPH nucleic acid molecules are provided. In this aspect, the isolated nucleic acid molecules are selected from the group that consists of (a) a fragment of the nucleotide sequence of the wild-type LIPH gene that is between 24 and 32 nucleotides in length or more, and (b) complements of (a).

The invention also provides isolated fragments of wild-type LIPH genes, mutant LIPH genes, and/or alleles of LIPH genes or complements thereof. Fragments can be used to identify related sequences, etc. Those of ordinary skill in the art may apply no more than routine procedures identify and prepare fragments of LIPH genes (wild-type, mutant, and/or to allelic).

Fragments can be used as probes in Southern blot, Northern blot, and Gene Chip/microarray assays to identify such nucleic acid molecules, or can be used in amplification assays such as those employing PCR. As known to those skilled in the art, large probes such as 200 nucleotides or more are preferred for certain uses such as Southern blots, while smaller fragments will be preferred for uses such as in PCR and gene chip/microarray assays. Fragments also can be used to produce fusion proteins for generating antibodies or determining binding of the polypeptide fragments, or for generating immunoassay components. Likewise, fragments can be employed to produce nonfused fragments of the LIPH polypeptides that are useful, for example, in the preparation of antibodies in immunoassays.

A fragment of a LIPH gene may retain some property of the larger nucleic acid molecule, such as coding for a functional or non-functional polypeptide, lipase activity or lack of lipase activity. One of ordinary skill in the art can readily determine using the assays described herein and those well known in the art to determine whether a fragment retains characteristics of the full-length nucleic acid molecule using no more than routine experimentation.

In yet another aspect of the invention, mutant LIPH nucleic acid molecules are provided, which do not encode fully functional LIPH polypeptides. Rather, these mutant LIPH nucleic acid molecules of the invention include a sequence of wild-type LIPH nucleic acid with the exception that the sequence includes one or more mutations, e.g., deletions, additions or substitutions, such that the mutant LIPH nucleic acid molecules encodes a mutant LIPH polypeptide, i.e., a polypeptide that does not exhibit 100% of wild-type LIPH polypeptide functional activity. It is understood that some mutants will encode non-functional LIPH polypeptides, and other mutants will encode LIPH polypeptides with reduced or enhanced function. For example, a mutant LIPH molecule may encode a LIPH polypeptide that has from 0 through 25% of LIPH polypeptide functional activity, 26% through 50% of LIPH functional activity, 51% through 75% of LIPH polypeptide functional activity, or 76% through 95% of LIPH polypeptide functional activity, as assessed, for example, by the activity of the LIPH polypeptide in the hair growth pathway and/or by assessment of hair growth in a cell with mutant polypeptide expression. It will be understood by one of ordinary skill in the art, that some mutant LIPH nucleic acids may encode polypeptides that have over 100% of LIPH polypeptide functional activity. For example, a mutant may encode a LIPH polypeptide that has from 101% through 125% of LIPH functional activity, 125% through 150% of LIPH functional activity, or 150% through 200% or more of LIPH functional activity as assessed for example, by assessment of LIPH activity in the hair growth pathway and/or by assessment of hair growth and/or maintenance. The level of function of a mutant LIPH polypeptide can be determined and compared to that of wild-type LIPH polypeptide using standard assays known to one of ordinary skill in the art. Such assays include, but are not limited to the assays described herein. As used herein, the term “modulate the function and or activity” means to either inhibit or enhance the function and/or activity, e.g. of an LIPH polypeptide in a hair growth pathway.

As used herein with respect to nucleic acid molecules, in general, the term “isolated” means: (i) amplified in vitro by, for example, PCR; (ii) recombinantly produced by cloning; (iii) purified, as by cleavage and gel separation; or (iv) synthesized by, for example, chemical synthesis. An isolated nucleic acid molecule is one which is readily manipulable by recombinant DNA techniques well known in the art. Thus, a nucleotide sequence contained in a vector in which 5′ and 3′ restriction sites are known or for which PCR primer sequences have been disclosed is considered isolated but a nucleic acid sequence existing in its native state in its natural host is not. An isolated nucleic acid molecule may be substantially purified, but need not be. For example, a nucleic acid molecule that is isolated within a cloning or expression vector is not pure in that it may comprise only a tiny percentage of the material in the cell in which it resides. Such a nucleic acid molecule is isolated, however, as the term is used herein because it is readily manipulable by standard techniques known to those of ordinary skill in the art. An isolated nucleic acid molecule as used herein is not a naturally occurring chromosome.

As used herein, a “mutant LIPH nucleic acid molecule” refers to a LIPH nucleic acid molecule which includes a mutation (addition, deletion, or substitution) such that the mutant LIPH nucleic acid molecule does not encode a fully functional LIPH polypeptide. Rather, the mutant LIPH nucleic acid molecule encodes a mutant LIPH polypeptide, i.e., a polypeptide that may or may not exhibit the same functional activity as an LIPH polypeptide. Thus, a “mutant LIPH polypeptide” refers to a gene product of a mutant LIPH nucleic acid molecule. Some LIPH mutant nucleic acids encode mutant LIPH polypeptides that lack the function of a wild-type LIPH polypeptide in a hair growth pathway of a follicle cell. As used herein, the term “abnormal” may refer to an expression level or to the sequence of the nucleic acid and/or encoded polypeptide. Thus abnormal expression may be decreased expression (including zero expression) of a wild-type LIPH molecule (nucleic acid or polypeptide) as compared to its expression in a subject who does not have an LIPH-associated hair growth disorder. Similarly, abnormal expression may be increased expression of a mutant LIPH molecule (nucleic acid or polypeptide) as compared to its expression in a subject who does not have an LIPH-associated hair growth disorder.

According to yet another aspect of the invention, an expression vector comprising any of the isolated nucleic acid molecules of the invention, preferably operably linked to a promoter is provided. In a related aspect, host cells transformed or transfected with such expression vectors also are provided. Vectors may be useful for delivery of a LIPH molecule (or other polypeptide associated with the hair growth pathway that comprises LIPH) of the invention to a cell and/or subject.

As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding polypeptides which increase or decrease either resistance or sensitivity to antibiotics or other compounds, genes which encode enzymes whose activities are detectable by standard assays known in the art (e.g., β-galactosidase, alkaline phosphatase, or luciferase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional polypeptide, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a polypeptide. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. Vectors of the invention may optionally include 5′ leader or signal sequences. The choice and design of an appropriate vector is within the ability and discretion of one of ordinary skill in the art.

It will also be recognized that the invention embraces the use of the LIPH cDNA sequences or mutant LIPH cDNA sequences in expression vectors, as well as to transfect host cells and cell lines, be these prokaryotic (e.g., E. coli), or eukaryotic (e.g., CHO cells, COS cells, HEK293 cells, Xenopus oocytes, yeast expression systems and recombinant baculovirus expression in insect cells). Especially useful are mammalian cells such as human, mouse, hamster, pig, goat, primate, etc. They may be of a wide variety of tissue types, including hair follical cells, neuronal cells, fibroblasts, oocytes, monocytes, lymphocytes, and they may be primary cells or cell lines. Specific examples include hair follicle cells, neuronal cells, and embryonic stem cells. The expression vectors require that the pertinent sequence, i.e., those nucleic acids described herein be operably linked to a promoter.

In some embodiments, LIPH-encoding DNA is ligated into a vector, and introduced into suitable host cells to produce transformed cell lines that with wild-type or altered hair growth. The resulting cell lines can then be produced in quantity for reproducible quantitative analysis of the effects of known or potential drugs on hair growth and/or maintenance. Transfected mammalian cells may then be used in the methods of drug and compound screening provided herein.

Eukaryotic cells in which DNA or RNA may be introduced include any cells that are transfectable by such DNA or RNA or into which such DNA or RNA may be injected. Preferred cells are those that can be transiently or stably transfected and also express the DNA and RNA. Cells for use in transfection methods of the invention may be identified empirically or selected from among those known to be readily transfected or injected.

Exemplary cells for introducing DNA include cells of mammalian origin (e.g., COS cells, mouse L cells, Chinese hamster ovary (CHO) cells, human embryonic kidney (HEK) cells (particularly HEK293 cells that can be frozen in liquid nitrogen and then thawed and regrown; for example, those described in U.S. Pat. No. 5,024,939 to Gorman (see, also, Stillman et al. (1985) Mol. Cell. Biol. 5:2051-2060)), African green monkey cells (and other such cells known to those of skill in the art), amphibian cells (e.g., Xenopus laevis oocytes), yeast cells (e.g., Saccharomyces cerevisiae, Pichia pastoris), and the like. Exemplary cells for expressing injected RNA transcripts include Xenopus laevis oocytes. Cells for transfection of DNA are known to those of skill in the art or may be empirically identified, and include HEK293 (which are available from ATCC under accession #CRL 1573); Ltk⁻ cells (which are available from ATCC under accession #CCL1.3); COS-7 cells (which are available from ATCC under accession #CRL 1651); and DG44 cells (dhfr⁻ CHO cells; see, e.g., Urlaub et al. (1986) Cell. Molec. Genet. 12:555).

DNA may be stably incorporated into cells or may be transiently expressed using methods known in the art. Stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene (such as the E. coli β-galactosidase gene) to monitor transfection efficiency. Selectable marker genes usually are not included in the transient transfections because the transfectants are typically not grown under selective conditions, and are usually analyzed within a few days after transfection.

To produce such stably or transiently transfected cells, the cells should be transfected with a sufficient concentration of LIPH-encoding nucleic acids to produce LIPH polypeptide encoded by heterologous DNA. The precise amounts and ratios of DNA encoding the subunits may be empirically determined and optimized for a particular combination of subunits, cells and assay conditions.

Heterologous DNA may be maintained in the cell as an episomal element or may be integrated into chromosomal DNA of the cell. The resulting recombinant cells may then be cultured or subcultured (or passaged, in the case of mammalian cells) from such a culture or a subculture thereof. Methods for transfection, injection and culturing recombinant cells are known to the skilled artisan.

As used herein, the terms “heterologous”, “exogenous”, or “foreign” DNA and RNA are used interchangeably and refer to DNA or RNA that does not occur naturally as part of the genome of the cell in which it is present or to DNA or RNA that is found in a location or locations in the genome that differ from that in which it occurs in nature. Typically, heterologous or foreign DNA and RNA refers to DNA or RNA that is not endogenous to the host cell and has been artificially introduced into the cell. Examples of heterologous DNA include DNA that encodes mutant LIPH polypeptide, DNA that encodes RNA or polypeptides that mediate or alter expression of endogenous DNA by affecting transcription, translation, or other regulatable biochemical processes, and the like. The cell that expresses heterologous DNA may contain DNA encoding the same or different expression products. Heterologous DNA need not be expressed and may be integrated into the host cell genome or maintained episomally.

In general, the conditional expression vectors used in such systems use a variety of promoters which confer the desired gene expression pattern (e.g., temporal or spatial). Conditional promoters also can be operably linked to LIPH nucleic acid molecules to increase or decrease expression of an LIPH molecule in a regulated or conditional manner.

The invention, in part, relates to constructs, transgenic cells, and transgenic cell lines that can be used to examine hair growth and diagnose hair growth disorders in a mammal. Constructs, transgenic cells, and transgenic cell lines can also be used to test compounds to identify compounds that increase hair growth. A DNA construct of the invention is also referred to herein as a genetic construct or a nucleic acid construct. Cells transfected with a genetic construct can be used in assays of hair growth. Constructs and cells of the invention may be designed to express a modified level of hair, as compared to a control and/or wild-type cell. Constructs, transgenic cells, and transgenic cell lines of the invention can be used to monitor and assess hair growth. Cells of tissues carrying the transgene can be cultured using standard tissue culture techniques, and used, e.g., to study the functioning of the transgenic cells.

Recombinant nucleic acid constructs that can be used to prepare transgenic cells or cell lines of the invention may include, at least, a promoter operably linked to a sequence encoding an LIPH polypeptide or other polypeptide involved in the hair growth pathway that comprises LIPH. Expression of the polypeptide can be driven by the activation of the promoter. In some embodiments, two or more different constructs may be used in a transgenic cell of the invention, each expressing a different polypeptide. In some embodiments of the invention, a fragment of the nucleic acid sequence that encodes an LIPH polypeptide or other peptide associated with the pathway for hair growth that comprises LIPH.

Methods for generating transgenic cells typically include the steps of (1) assembling a suitable DNA construct useful for inserting a specific DNA sequence into the nuclear genome of a cell; (2) transfecting the DNA construct into the cells; (3) allowing random insertion and/or homologous recombination to occur. The modification resulting from this process may be the insertion of a suitable DNA construct(s) into the target genome; deletion of DNA from the target genome; and/or mutation of the target genome.

Genetic constructs (e.g., plasmids) may include a gene of interest as well as a variety of elements including one or more regulatory promoters, insulators, enhancers, and repressors as well as elements for ribosomal binding to the RNA transcribed from the DNA construct. Constructs also may have suitable origins of replication and/or selectable markers. These and other examples are well known to those of ordinary skill in the art and are not meant to be limiting.

Due to the effective recombinant DNA techniques available in conjunction with DNA sequences for regulatory elements and genes readily available in databases and the commercial sector, one of ordinary skill in the art can readily generate a DNA construct appropriate for establishing transgenic cells using the materials and methods described herein.

Transfection techniques are well known to those of ordinary skill in the art and materials and methods for carrying out transfection of DNA constructs into cells are commercially available. For example, materials that can be used to transfect cells with DNA constructs are lipophilic compounds such as Lipofectin™, activated polycationic dendrimers such as Superfect™, LipoTAXI™, and CLONfectin™. Particular lipophilic compounds can be induced to form liposomes for mediating transfection of the DNA construct into the cells. In addition, cationic based transfection agents that are known in the art can be utilized to transfect cells with nucleic acid molecules (e.g., calcium phosphate precipitation). Also, electroporation techniques known in the art can be utilized to translocate nucleic acid molecules into cells. Furthermore, particle bombardment techniques known in the art can be utilized to introduce exogenous DNA into cells. Target sequences from a DNA construct can be inserted into specific regions of the nuclear genome by rational design of the DNA construct. These design techniques and methods are well known to a person of ordinary skill in the art. See, for example, U.S. Pat. No. 5,633,067; U.S. Pat. No. 5,612,205; and PCT publication WO 93/22432, each of which is incorporated herein by reference in its entirety. Once the desired DNA sequence is inserted into the nuclear genome of a cell, the location of the insertion region as well as the frequency with which the desired DNA sequence has inserted into the nuclear genome can be identified by methods well known to those of ordinary skill in the art.

Constructs, transgenic cells, and transgenic cell lines of the invention can be used in methods to assess candidate compounds and/or treatments for hair growth disorders and/or conditions and to assess levels and activity of LIPH nucleic acids and polypeptides. For example, candidate pharmaceutical agents or compounds may be tested using transgenic cells to determine whether the agent or compound modulates (inhibits or enhances) hair growth. A candidate compound can also be tested using transgenic cells or cell lines to determine a therapeutically effective amount of the compound, which is that amount effective to modulate hair growth. In some embodiments, a preferred agent is one that enhances hair growth. Additional tests useful for monitoring the onset, progression, and/or remission, of hair growth associated with a disease or condition such as those described herein, can be performed using transgenic cells and/or cell lines. In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition.

Levels of hair growth and/or activity of LIPH and/or other peptides associated with the pathway for hair growth that comprises LIPH in a transgenic cell can be determined using methods described herein to assess the level of a reporter protein and are advantageously compared to controls according to the invention. Healthy normal cells may be transgenic normal cells.

Kits containing a construct, transgenic cell and/or transgenic cell line of the invention can be prepared for in vivo, ex vivo, and/or, in vitro diagnosis, prognosis and/or monitoring hair growth-associated disease or condition using any suitable method known in the art. The components of the kits may be packaged frozen, chilled, or at room temperature and certain components may be packaged in aqueous medium or in lyophilized form.

A kit may comprise a carrier being compartmentalized to receive in close confinement therein one or more container means or series of container means such as test tubes, vials, flasks, bottles, syringes, cages, or the like. A first of said container means or series of container means may contain one or more constructs, or transgenic cells of the invention. A kit of the invention may also include a control. The kit also may further comprise instructions as described above. The instructions typically will be in written form and will provide guidance for carrying out the assays embodied by the kit and for making a determination based upon that assay.

The invention also embraces so-called expression kits, which allow the artisan to prepare a desired expression vector or vectors. Such expression kits include at least separate portions of each of the previously discussed coding sequences. Other components may be added, as desired, as long as the previously mentioned sequences, which are required, are included.

According to another aspect of the invention, an isolated LIPH polypeptide encoded by any of the foregoing isolated nucleic acid molecules of the invention is provided. As used herein, an LIPH polypeptide refers to a polypeptide that is encoded by a wild-type LIPH nucleic acid or a mutant LIPH nucleic acid, or a functional equivalent thereof, provided that the functional equivalent encodes an LIPH polypeptide which exhibits an activity of a wild-type or mutant LIPH activity. As used herein, an LIPH functional activity refers to the activity of an LIPH polypeptide in a pathway of hair production in a hair follicle cells. An LIPH functional activity may also refer to the lipase function of an LIPH polypeptide. In some embodiments of the invention a mutant LIPH polypeptide has reduced or no lipase function as compared to a wild-type LIPH polypeptide.

In some embodiments an isolated polypeptide comprises the amino acid sequence of a wild-type LIPH polypeptide or a fragment thereof. In some embodiments, an isolated polypeptide of the invention may comprise the amino acid sequence of a mutant LIPH polypeptide or a fragment thereof that has reduced or absent LIPH functional activity. Such polypeptides are useful, for example, alone or as fusion polypeptides to generate antibodies for LIPH polypeptides. In addition, expression and/or function of the LIPH polypeptides of the invention can be modulated using methods described herein and the effect on hair growth can be evaluated as a method of determining the process in normal and abnormal hair growth and disorders.

LIPH polypeptides of the invention can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed polypeptide. Short polypeptides, including antigenic peptides (such as those presented by MHC molecules on the surface of a cell for immune recognition) also can be synthesized chemically using well-established methods of peptide synthesis.

Thus, as used herein with respect to polypeptides, “isolated” means separated from its native environment and present in sufficient quantity to permit its identification or use. Isolated, when referring to a protein or polypeptide, means, for example: (i) selectively produced by expression of a recombinant nucleic acid or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated polypeptide may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in living systems, e.g. isolated from other polypeptides.

A fragment of an LIPH polypeptide, for example, generally has the features and characteristics of fragments as discussed above in connection with nucleic acid molecules. Fragments of LIPH polypeptides may be any length shorter than a full-length LIPH polypeptide and in some embodiments of the invention may be short segments, typically between 5 and 14 amino acids (e.g., 5, 6, 7, 8, 9, 10, 11, 12, 13, and 14 amino acids long). In some embodiments, fragments of LIPH polypeptides may be used to generate LIPH-specific antibodies.

Fragments of a polypeptide may be fragments that retain a distinct functional capability of the polypeptide. Functional capabilities which can be retained in a fragment of an LIPH polypeptide include interaction with antibodies, interaction with other polypeptides or fragments thereof, function in a pathway of hair growth in a follicle cell, selective binding of nucleic acid molecules, and lipase activity. An LIPH polypeptide can be assessed with respect to the identity of fragments of the polypeptide that may function in a hair growth pathway in a hair follicle cell. For example, fragments can be tested in a cell or animal determine the size and sequence of fragments that when expressed allow normal or abnormal hair growth. One activity of a LIPH polypeptide is the ability to provoke an immune response to a mutant LIPH molecule but not provoke an immune response to normal levels of a nonmutated LIPH molecule. For example, identification of a fragment of a mutant LIPH polypeptide that is antigenic, in contrast to a non-antigenic normal LIPH polypeptide.

Those skilled in the art are well versed in methods for selecting unique amino acid sequences, typically on the basis of the ability of the fragment to selectively distinguish the sequence of interest from non-family members. A comparison of the sequence of the fragment to those on known databases typically is all that is necessary.

The invention embraces variants of the LIPH polypeptides described herein. As used herein, a “variant” of an LIPH polypeptide is a polypeptide which contains one or more modifications to the primary amino acid sequence of an LIPH polypeptide. Thus, a variant LIPH polypeptide may be a mutant LIPH polypeptide. Modifications which create an LIPH polypeptide variant can be made to an LIPH polypeptide to 1) increase, reduce, or eliminate an activity of the LIPH polypeptide; 2) enhance a property of the LIPH polypeptide, such as polypeptide stability in an expression system or the stability of protein-protein binding; or 3) provide a novel activity or property to an LIPH polypeptide, such as addition of an antigenic epitope or addition of a detectable moiety.

Modifications to an LIPH polypeptide are typically made to the nucleic acid molecule which encodes the polypeptide, and can include deletions, point mutations, truncations, amino acid substitutions, and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the LIPH amino acid sequences. One of skill in the art will be familiar with methods for predicting the effect on polypeptide conformation of a change in polypeptide sequence, and can thus “design” a variant LIPH polypeptide according to known methods. One example of such a method is described by Dahiyat and Mayo in Science 278:82-87, 1997, whereby polypeptides can be designed de novo. The method can be applied to a known polypeptide to vary only a portion of the polypeptide sequence. By applying the computational methods of Dahiyat and Mayo, specific variants of an LIPH polypeptide can be proposed and tested to determine whether the variant retains a desired conformation.

In general, variants include LIPH polypeptides that are modified to alter their function (e.g., to reduce and/or eliminate lipase activity) an also may include LIPH polypeptides that are modified specifically to alter a feature of the polypeptide unrelated to its desired physiological activity. For example, cysteine residues can be substituted or deleted to prevent unwanted disulfide linkages. Similarly, certain amino acids can be changed to enhance expression of an LIPH polypeptide by eliminating proteolysis by proteases in an expression system (e.g., dibasic amino acid residues in yeast expression systems in which KEX2 protease activity is present).

Mutations of a nucleic acid molecule that encodes an LIPH polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant LIPH polypeptides) that are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of an LIPH gene or cDNA clone to enhance or inhibit expression of the polypeptide. The activity of variants of LIPH polypeptides can be tested by cloning the gene encoding the variant LIPH polypeptide into a bacterial, amphibian, or mammalian expression vector, introducing the vector into an appropriate host cell, expressing the variant LIPH polypeptide, and testing for a functional capability of the LIPH polypeptide as disclosed herein. Preparation of other variant polypeptides may favor testing of other activities, as will be known to one of ordinary skill in the art.

The skilled artisan will also realize that conservative amino acid substitutions may be made in LIPH polypeptides to provide functional variants of the foregoing polypeptides, i.e, the variants which the functional capabilities of the LIPH polypeptides. As used herein, a “conservative amino acid substitution” refers to an amino acid substitution which does not to alter the relative charge or size characteristics of the polypeptide in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a polypeptide derived from an LIPH polypeptide possesses a reduced LIPH activity one can make conservative amino acid substitutions to the amino acid sequence of the polypeptide or can be made through deletions of one or more amino acids from the LIPH sequence. The substituted polypeptides or polypeptides with deletions can then be tested for one or more of the above-noted functions, in vivo or in vitro. These variants can be tested for improved stability and are useful, inter alia, in pharmaceutical compositions.

Variants of LIPH polypeptides that retain or lose the function of LIPH polypeptides, can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary variants of the LIPH polypeptides, although not intended to be limiting, include substitutions of the polypeptides set forth as SEQ ID NO:1 or mutant LIPH polypeptides and/or LIPH polypeptides with one or more amino acid deletions Amino acid substitutions or deletions in the amino acid sequence of LIPH polypeptides to produce functional and reduced-function variants of LIPH polypeptides typically are made by alteration of the nucleic acid molecule encoding an LIPH polypeptide. Non-limiting examples of amino acids of a LIPH polypeptide that may be substituted or deleted to reduce lipase function of a LIPH polypeptide are amino acids encoded by exon 4 of the nucleic acid that encodes LIPH polypeptide, and amino acids Ser-154, Asp-178, and/or His-248 of an LIPH amino acid sequence (e.g., SEQ ID NO:1). These and other substitutions and/or deletions can be made by a variety of methods known to one of ordinary skill in the art. For example, amino acid substitutions or deletions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding an LIPH polypeptide. Where amino acid substitutions or deletions are made to a small fragment of an LIPH polypeptide, the substitutions can be made by directly synthesizing the polypeptide. The activity of functional variants or fragments of LIPH polypeptide can be tested by cloning the gene or transcript that encodes the altered LIPH polypeptide into a bacterial, mammalian, or insect cell expression vector, introducing the vector into an appropriate host cell, expressing the altered LIPH polypeptide, and testing for a functional capability of the LIPH polypeptides as disclosed herein.

The invention as described herein has a number of uses, some of which are described elsewhere herein. First, the invention permits isolation of LIPH polypeptide molecules. A variety of methodologies well known to the skilled practitioner can be utilized to obtain isolated LIPH molecules. The polypeptide may be purified from cells that naturally produce the polypeptide by chromatographic means or immunological recognition. Alternatively, an expression vector may be introduced into cells to cause production of the polypeptide. In another method, mRNA transcripts may be microinjected or otherwise introduced into cells to cause production of the encoded polypeptide, as described herein. Translation of mRNA in cell-free extracts such as the reticulocyte lysate system also may be used to produce polypeptide. Those skilled in the art also can readily follow known methods for isolating LIPH polypeptides. These include, but are not limited to, HPLC, size-exclusion chromatography, ion-exchange chromatography, and immune-affinity chromatography. In addition, those skilled in the art will also be able to utilize recombinant LIPH to determine the structure (e.g. quaternary, tertiary) of an LIPH polypeptide or its variants.

The isolation and identification of LIPH nucleic acid molecules and polypeptides also makes it possible for the artisan to diagnose a disorder characterized by abnormal expression or function of an LIPH nucleic acid molecule or polypeptide. These methods involve determining the abnormal expression of one or more LIPH nucleic acid molecules and/or encoded LIPH polypeptides. In the former situations, such determinations can be carried out via any standard nucleic acid determination assay, including the polymerase chain reaction (PCR), or assaying with labeled hybridization probes, etc. In the latter situations, such determinations can be carried out by assaying biological samples with binding partners (e.g., antibodies) for LIPH polypeptides.

The invention provides agents which bind to LIPH polypeptides encoded by LIPH nucleic acid molecules, respectively, and in certain embodiments preferably to unique fragments of the LIPH polypeptides. Such binding partners can be used in screening assays to detect the presence or absence of a LIPH polypeptide and in purification protocols to isolate such LIPH polypeptides. Likewise, such binding partners can be used to selectively target drugs, toxins or other molecules to cells which express mutant LIPH polypeptides. In this manner, cells present in tissues that express mutant LIPH polypeptides can be treated with therapeutic compounds. Such agents also can be used to inhibit the native activity of the LIPH polypeptides, for example, by binding to such polypeptides, to further characterize the functions of these molecules.

In addition, antibodies generated against the above-described LIPH and mutant LIPH polypeptides may be employed for studying tissue localization, structure of functional domains, as well as in diagnostic applications, therapeutic applications, and the like.

Factors to consider in selecting portions of the LIPH polypeptide for use as an immunogen (as either a synthetic peptide or a recombinantly produced bacterial fusion protein) include antigenicity, accessibility (i.e., extracellular and cytoplasmic domains), uniqueness to the particular subunit, etc.

The availability of mutant-specific antibodies makes possible the application of the technique of immunohistochemistry to monitor the distribution and expression density of mutant LIPH (e.g., in normal versus diseased follicle cell). Such antibodies could also be employed for diagnostic and therapeutic applications. For example, invention provides methods for modulating hair growth by contacting LIPH polypeptides with an effective amount of an antibody.

The invention, therefore, provides antibodies or fragments of antibodies having the ability to selectively bind to LIPH polypeptides, and preferably to mutant LIPH polypeptides. Antibodies include polyclonal, monoclonal, and chimeric antibodies, prepared, e.g., according to conventional methodology.

As detailed herein, the foregoing antibodies and other binding molecules may be used for example to identify tissues expressing LIPH polypeptide or to purify LIPH polypeptide. Antibodies and LIPH polypeptides of the invention may be coupled to specific detectable labels for imaging LIPH expression in cells and tissues, and coupled to therapeutically useful agents according to standard coupling procedures. A wide variety of detectable labels are available for use in methods of the invention and may include labels that provide direct detection (e.g., fluorescence, colorimetric, or optical, etc.) or indirect detection (e.g., enzyme-generated luminescence, epitope tag such as the FLAG epitope, enzyme tag such as horseradish peroxidase, labeled antibody, etc.). A variety of methods may be used to detect a detectable label depending on the nature of the label and other assay components. Labels may be directly detected through optical or electron density, radioactive emissions, nonradiative energy transfers, etc. or indirectly detected with antibody conjugates, strepavidin-biotin conjugates, etc. Methods for using and detecting labels are well known to those of ordinary skill in the art. Methods of the invention may be used for in vivo, in vitro, and/or ex vivo imaging, including but not limited to real-time imaging. The presence of a labeled antibody in a subject can be detected by in vivo, ex vivo, or in vitro imaging using standard methods. Examples of detection methods include, but are not limited to, immunohistochemistry, Western blot of tissues or cells, or by any other suitable detection method.

The term “detectable label” as used here means a molecule preferably selected from, but not limited to, fluorescent, enzyme, radioactive, metallic, biotin, chemiluminescent, and bioluminescent molecules. As used herein, a detectable label may be a colorimetric label, e.g., a chromophore molecule. In some aspects of the invention, a polypeptide or an antibody may be detectably labeled with a single or with two or more of the detectable labels set forth herein, or other art-known detectable labels.

Radioactive or isotopic labels may be, for example, ¹⁴C, ³H, ³⁵S, ¹²⁵I, and ³²P. Fluorescent labels may be any compound that emits an electromagnetic radiation, preferably visible light, resulting from the absorption of incident radiation and persisting as long as the stimulating radiation is continued.

Examples of fluorescent labels that may be used on polypeptides and/or antibodies of the invention and in methods of the invention include but are not limited to 2,4-dinitrophenyl, acridine, cascade blue, rhodamine, 4-benzoylphenyl, 7-nitrobenz-2-oxa-1,3-diazole, 4,4-difluoro-4-bora-3a,4a-diaza-3-indacene and fluorescamine. Absorbance-based labels may be molecules that are detectable by the level of absorption of various electromagnetic radiation. Such molecules may be, for example, the fluorescent labels indicated above.

Chemiluminescent labels in this invention refer to compounds that emit light as a result of a non-enzymatic chemical reaction. Methods of the invention may also include the use of a luminescent detectable diagnostic molecule such as enhanced green fluorescent protein (EGFP), luciferase (Luc), or another detectable expression product.

Enzymatic methods for detection may be used including the use of alkaline phosphatase and peroxidase. Additional enzymes may also be used for detection in methods and kits of the invention.

As used herein, fluorophores include, but are not limited to amine-reactive fluorophores that cover the entire visible and near-infrared spectrum. Examples of such fluorophores include, but are not limited to, 4-methylumbelliferyl phosphate, fluorescein isothiocyanate (FITC), tetramethylrhodamine isothiocyanate (TRITC), BODIPY dyes; Oregon Green, rhodamine green dyes; the red-fluorescent Rhodamine Red-X, Texas Red dyes; and the UV light—excitable Cascade Blue, Cascade Yellow, Marina Blue, Pacific Blue and AMCA-X fluorophores. Fluorophores may also include non-fluorescent dyes used in fluorescence resonance energy transfer (FRET).

A labeled polypeptide or antibody of the invention can be prepared from standard moieties known in the art. As is recognized by one of ordinary skill in the art, the labeling process for preparing a detectable labeled antibody or fragment thereof may vary according to the molecular structure of the antibody and the detectable label. Methods of labeling polypeptides and/or antibodies with one or more types of detectable labels are routinely used and are well understood by those of ordinary skill in the art.

In some embodiments, it is contemplated that one may wish to first derivatize a polypeptide or antibody, and then attach the detectable label to the derivatized product. Suitable cross-linking agents for use in this manner include, for example, SPDP (N-succinimidyl-3-(2-pyridyldithio)propionate), and SMPT, 4-succinimidyl-oxycarbonyl-methyl-(2-pyridyldithio)toluene. In some embodiments, a radionuclide may be coupled to an antibody or antigen-binding fragment thereof by chelation. Other diagnostic and/or detectable labeling agents useful in the invention will be apparent to one of ordinary skill in the art.

According to a further aspect of the invention, pharmaceutical compositions containing the nucleic acid molecules, polypeptides, and/or binding polypeptides of the invention are provided. Pharmaceutical compositions containing one or more compounds to modulate hair growth such as a simple lipid (e.g., a lysophosphatidic acid (LPA), 1- or 2-acyl-lysophosphatidic acid, or a modified LPA) or other compound identified using methods of the invention, are also provided. A composition of the invention may contain any of the foregoing therapeutic molecules, polypeptides, and/or compounds in a carrier. In some aspects, a carrier may be a pharmaceutically acceptable carrier. Thus, in a related aspect, the invention provides a method for forming a medicament that involves placing a therapeutically effective amount of the therapeutic agent in a carrier to form one or more doses.

When administered, a therapeutic composition of the present invention may be administered in a pharmaceutically acceptable preparation. Such preparations may routinely contain pharmaceutically acceptable concentrations of salt, buffering agents, preservatives, compatible carriers, and optionally other therapeutic agents.

A carrier useful in a composition of the invention may be a carrier suitable for topical application to the skin within which the essential materials (e.g., an active ingredient or compound) and optional other materials are incorporated to enable the essential materials and optional components to be delivered to the hair follicle at an appropriate concentration. A carrier can thus act as a diluent, dispersant, solvent, or the like for the various components of the compositions of the invention including particulate material(s) and the actives that ensure that they can be applied to and distributed evenly over the selected target at an appropriate concentration. A composition for topical administration may also include one or more compounds that act as skin softeners, skin permeator, or as agents that enable the active ingredient of the composition to access the hair follicle. A composition for topical administration may also include one or more penetrants appropriate to the barrier to be permeated, e.g. appropriate to allow access of the active ingredient of the composition to the hair follicle.

A carrier can be solid, semi-solid or liquid. In some embodiments a carrier is a liquid or semi-solid, such as a cream, lotion or gel. In some embodiments a carrier is in the form of a lotion, cream, or a gel, that has a sufficient thickness or yield point to prevent the particles from sedimenting. A carrier may itself be inert or it can possess dermatological benefits of its own. A carrier should also be physically and, chemically compatible with the essential components described herein, and should not unduly impair stability, efficacy, or other use benefits associated with the compositions of the invention. In some embodiments, active components are micronized for inclusion into the compositions for enhanced activity.

The type of carrier utilized in compositions of the invention depends on the type of product form desired for the composition. Topical compositions useful in the subject invention may be made into a wide variety of product forms such as are known in the art. These include, but are not limited to, lotions, creams, gels, sticks, sprays, ointments, pastes, and mousses. Such product forms may comprise several types of carriers including, but not limited to, solutions, aerosols, emulsions, gels, solids, and liposomes. Preferred carriers contain a dermatologically acceptable, hydrophilic diluent. Suitable hydrophilic diluents include water, organic hydrophilic diluents such as C₁-C₄ monohydric alcohols and low molecular weight glycols and polyols, including propylene glycol, polyethylene glycol (e.g. of MW 200-600), polypropylene glycol (e.g. of MW 425-2025), glycerol, butylene glycol, 1,2,4-butanetriol, sorbitol esters, 1,2,6-hexanetriol, ethanol, iso-propanol, sorbitol esters, ethoxylated ethers, propoxylated ethers and combinations thereof. A diluent in some embodiments may be liquid. In some embodiments, water is used as a diluent. A composition may comprise at least about 40%, 50%, 60%, 70%, or more of the hydrophilic diluent.

In some embodiments a carrier comprises an emulsion comprising a hydrophilic phase, especially an aqueous phase, and a hydrophobic phase e.g., a lipid, oil, or oily material. As known to one of ordinary skill in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one of ordinary skill in the art which means that the phase exists as small particles or droplets that are suspended in and surrounded by a continuous phase. A dispersed phase is also known as an internal or discontinuous phase. An emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% (preferably about 1% to about 30%) of the dispersed hydrophobic phase and from about 1% to about 99% (preferably from about 40% to about 90%) of the continuous hydrophilic phase; water-in-oil emulsions typically comprise from about 1% to about 98% (preferably from about 40% to about 90%) of the dispersed hydrophilic phase and from about 1% to about 50% (preferably about 1% to about 30%) of the continuous hydrophobic phase. An emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92, incorporated herein by reference. In some embodiments, compositions herein are oil-in-water emulsions. A composition of the invention may be administered with an agent or compound that enhances transdermal delivery of the therapeutic compound.

A composition of the present invention for topical administration may be formulated, marketed, and/or used in conjunction with shampoos, conditioners, styling gels, or other hair care products or in lotions, creams, emulsions, gels, or other skin care products. For example, a composition of the invention may be administered in conjunction with a shampoo and/or a conditioner that improves the appearance or apparent thickness of hair. Non-limiting examples of additional components that may be administered with a composition of the invention include, but are not limited to plant extracts.

A compound of the invention to modulate hair growth may be administered alone or in conjunction with one or more additional compounds or therapeutic agents for the modulation of hair growth.

The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. Alternative carriers to those described above herein for topical application may be used in methods and compositions of the invention. The characteristics of a carrier will depend on the route of administration. Physiologically and pharmaceutically acceptable carriers include diluents, fillers, salts, buffers, stabilizers, solubilizers, and other materials which are well known in the art.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712). Those of skill in the art can readily determine the various parameters and conditions for producing antibody aerosols without resort to undue experimentation. When using antisense preparations of the invention, slow intravenous administration is preferred.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The preparations of the invention are administered in effective amounts. An effective amount is that amount of a pharmaceutical preparation that alone, or together with further doses, stimulates the desired response. In the case of treating a hair growth disorder, the desired response is modulating the amount of hair growth and/or maintenance in the subject. This may involve only slowing the progression of the disorder temporarily, although more preferably, it involves halting the progression of the disorder permanently. These responses can be monitored by routine methods or can be monitored according to diagnostic methods of the invention discussed herein. Typically effective amounts of a composition of the invention will be determined in clinical trials, establishing an effective dose for a test population versus a control population in a blind study.

The therapeutically effective amount of an LIPH molecule or other compound is that amount effective to modulate LIPH functional activity levels and reduce, prevent, delay onset, or eliminate the LIPH-associated hair loss disorder, or to modulate normal hair growth to a desired level of hair growth (e.g., affect a hair growth condition). For example, testing can be performed to determine the LIPH functional activity in a subject's tissue and/or cells. Additional tests useful for monitoring the onset, progression, and/or remission, of an LIPH-associated hair growth disorder or condition known to one of ordinary skill in the art. As would be understood by one of ordinary skill, for some hair loss disorders an effective amount would be the amount of LIPH molecules or other compound that increases or decreases LIPH functional activity in the pathway of hair growth in a hair follicle cell, to a level that diminishes the disorder, as determined by the aforementioned tests. It is also understood that in a normal subject desiring increased or decreases hair growth in one or more body regions an effective amount would be that amount of LIPH molecule or other compound that provides the desired hair growth level in that region.

The LIPH molecule or other compound dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

According to another aspect of the invention, various diagnostic methods are provided. In general, the methods are for diagnosing a hair growth disorder characterized by abnormal expression of an LIPH molecule. As used herein, “abnormal expression” is dependent upon the particular hair growth disorder (i.e., characterized by increased or decreased LIPH molecule expression, or an increase in expression of a mutant LIPH molecule). Thus “abnormal expression” refers to increased expression of normal LIPH, decreased expression (including no expression) of an LIPH molecule (nucleic acid or polypeptide), or increased expression of a “mutant LIPH molecule”. A mutant LIPH molecule refers to an LIPH nucleic acid molecule that includes a mutation (deletion, addition, or substitution) or to an LIPH polypeptide molecule (e.g., gene product of mutant LIPH nucleic acid molecule) that includes a mutation, provided that the mutation results in a mutant LIPH polypeptide that has reduced or no functional activity as compared to a non-mutant (e.g., wild-type) LIPH polypeptide. Diagnostic methods of the invention can be used to detect the presence of a disorder associated with abnormal expression and or function of an LIPH molecule, as well as to assess the progression and/or regression of the disorder such as in response to treatment (e.g., chemical therapy).

Expression of LIPH can be evaluated using standard methods known to those of ordinary skill in the art. Such methods include, but are not limited to: PCR, RT-PCT and antibody methods, which can be used to evaluate changes/alternations (for example, relative to normal) in the expression of LIPH at the gene, transcript, and polypeptide levels, respectively. The above-mentioned methods can be used to evaluate expression of LIPH molecule, whether or not it has normal functional activity. For example, an LIPH polypeptide may be expressed but lack normal function due to mutation that does not effect expression. The above-mentioned methods can be used to evaluate LIPH expression regardless of functional activity.

Expression of LIPH can also be evaluated by determining the functional activity of LIPH in cells and tissues with methods including, but not limited to, art-known assay methods and assays described in the Examples section. According to this aspect of the invention, the method for diagnosing a disorder characterized by abnormal expression of an LIPH molecule involves: detecting in a first biological sample obtained from a subject, expression of an LIPH molecule, wherein decreased or increased expression of the LIPH molecule (depending upon the disorder as discussed herein) compared to a control sample indicates that the subject has a hair growth disorder characterized by abnormal expression of an LIPH molecule. As used herein, an LIPH-associated hair growth disorder is a disorder in which an abnormality in expression or function of LIPH is associated with abnormal hair growth. Such an abnormality of expression or function may be due to a mutation in an LIPH gene or may be associated with a defect or abnormality in a pathway associated with LIPH. As used herein, the phrase “alternation in an LIPH gene” means a change from the wild-type sequence of the gene or a change in expression from that of a wild-type gene. In some embodiments an alteration of a gene may be a mutation, e.g. a deletion, substitution, addition. In certain embodiments, an alteration in a gene may be a reduction of expression.

As used herein, a “hair growth disorder characterized by abnormal expression of an LIPH molecule” refers to a hair growth disorder in which there is a detectable difference in the expression and/or function of LIPH molecule(s) in selected cells of a subject compared to the control levels of these molecules. Thus, a disorder characterized by abnormal expression of an LIPH molecule embraces overexpression of LIPH, under-expression of LIPH as well as expression of a mutant LIPH compared to control levels of these molecules. Such differences in expression and/or function levels can be determined in accordance with the diagnostic methods of the invention as disclosed herein. Disorders that are characterized by abnormal expression of an LIPH molecule include: hair growth disorders that are characterized by abnormally high and abnormally low levels of hair growth in a subject. Exemplary LIPH-associated hair growth disorders include, but are not limited to hypotrichosis, alopecia, alopecia areata, androgenetic alopecia, anagen effluvium, self-induced hair loss, telogen effluvium, scarring alopecia, aging, drug- or chemical-induced hair loss (e.g., resulting from chemotherapy agents, etc.). In certain embodiments, methods of the invention are useful to diagnose a LIPH-associated hair growth disorder.

In certain embodiments of the invention, a diagnostic method of the invention detects an LIPH molecule that is an LIPH nucleic acid molecule. In yet other embodiments, the methods involve detecting an LIPH polypeptide.

Various detection methods can be used to practice the diagnostic methods of the invention. For example, the methods can involve contacting a biological sample with an agent that selectively binds to LIPH molecules to detect these molecules. In certain embodiments, the LIPH molecule is a nucleic acid and the method involves using an agent that selectively binds to the LIPH molecule, e.g., a nucleic acid that hybridizes to the LIPH nucleic acid under high stringency conditions. In yet other embodiments, the LIPH molecule is a polypeptide and the method involves using an agent that selectively binds to the LIPH molecule, e.g., a binding polypeptide, such as an antibody, that selectively binds to the LIPH polypeptide.

According to still another aspect of the invention, kits for performing the diagnostic methods of the invention are provided. The kits include nucleic acid-based kits or polypeptide-based kits. According to the former embodiment, the kits may include: one or more nucleic acid molecules that hybridize to an LIPH nucleic acid molecule under high stringency conditions; and instructions for the use of the nucleic acid molecules in the diagnosis of a hair growth disorder associated with abnormal expression of an LIPH molecule. Nucleic acid-based kits optionally further include a first primer and a second primer, wherein the first primer and the second primer are constructed and arranged to selectively amplify at least a portion of an isolated LIPH nucleic acid molecule (e.g., a wild-type, an allelic, or a mutant LIPH nucleic acid molecule). Alternatively, polypeptide based-kits are provided. Such kits may include: one or more binding polypeptides that selectively bind to an LIPH polypeptide and instructions for the use of the binding polypeptides in the diagnosis of a disorder associated with abnormal expression of an LIPH molecule. In some embodiments, the binding polypeptides are antibodies or antigen-binding fragments thereof, such as those described above. In these and other embodiments, certain of the binding polypeptides bind to the mutant LIPH polypeptide but do not bind to the non-mutant LIPH polypeptide to further distinguish the expression of these polypeptides in a biological sample.

The foregoing kits may include instructions or other printed material on how to use the various components of the kits for diagnostic purposes.

The biological sample can be located in vivo or in vitro. For example, the biological sample can be a tissue in vivo and the agent specific for the LIPH nucleic acid molecule or polypeptide can be used to detect the presence of such molecules in the tissue (e.g., for imaging portions of the tissue that express the LIPH gene products). Alternatively, the biological sample can be located in vitro (e.g., a hair follicle sample, biopsy, tissue extract). In a particularly preferred embodiment, the biological sample can be a cell-containing sample, more preferably a sample containing hair follicle cells. Samples of tissue and/or cells for use in the various methods described herein can be obtained through standard methods. Samples can be surgical samples of any type of tissue or body fluid. Samples can be used directly or processed to facilitate analysis (e.g., paraffin embedding). Samples also can be cultured cells, tissues, or organs. Samples can also be used to identify mutations and to assist in the decision of a treatment of a hair loss condition. For example, the determination that a hair loss condition is a genetic condition, e.g. associated with a mutation in an LIPH gene or a deficiency in an LIPH pathway, can be used in the determination of an appropriate treatment for a subject with such a genetic condition. Thus, the identification of a mutation in exon 4 of a subject with a hair loss condition permits the selection of a treatment to increase LIPH activity in the subject.

In some subjects, with or without a hair growth disorder, it may be desirable to increase LIPH expression and/or functional activity, such as by administering a drug that increases LIPH activity in cells and tissues of a subject. Such a modulation in activity can be brought about by, for example, increasing expression of LIPH, and/or increasing the level of a mutant LIPH that has an increased level of activity as compared to the activity level of normal LIPH. In some embodiments, it may be desirable to increase the amount of simple lipids in a cell and/or subject to treat a hair growth disorder.

In general, the treatment methods involve administering an agent to modulate expression or activity of an LIPH molecule in a follicle cell. In certain embodiments, the method for treating a subject with a hair growth disorder characterized by abnormal expression of an LIPH molecule, involves administering to the subject an effective amount of an LIPH nucleic acid molecule to treat the disorder.

An alternative method for treating a subject with a disorder characterized by abnormal expression of an LIPH molecule, or treating a subject desirous of increased hair growth, may involve administering to the subject an effective amount of a compound that increases the level of a simple lipid in a hair follicle cell to treat the disorder. In some embodiments, the compound is a simple lipid. In some embodiments the simple lipid is a lysophosphatidic acid (LPA), in certain embodiments, the LPA is 1- or 2-acyl-lysophosphatidic acid, in certain embodiments the simple lipid is a modified LPA.

In some aspects the invention also may include additional compositions and methods for treatment of a hair growth disorder or condition. Therapeutic compositions of the invention may include the compounds that modulate (increase or decrease) the activity of molecules regulating upstream or downstream of pathway of LIPH. Examples of such molecules, though not intended to be limiting are 1) molecules that generate PA, 2) molecule precursors for phospholipases that generate different LPA or 3) inhibitors of enzymes that degrade PA or LPA. Molecules that modulate upstream or downstream actions of the LIPH pathway involved in hair growth may also be utilized in compositions and methods to treat a hair growth disorder.

In addition, the invention also includes, in some aspects the use of proteins homologous to LIPH such as PS-PLA1, LIPI or mPA-PLA1β, and/or other enzymes with similar activities (e.g., activities such as generating various LPAs). Such homologs may be administered to a subject to enhance hair growth. Types of phospholipases that can be utilized in methods and compositions of the invention may include mammalian, plant, or bacterial phospholipases. Administration of these types of enzymes (e.g., surface application) may be used to treat hair growth disorders. Such enzymes may be obtained from various sources, including, but not limited to fluids and/or tissues of mammals or lower organisms, including, but not limited to other vertebrates, invertebrates, and bacteria. In addition to naturally occurring LPAs and other simple lipids, it is also contemplated that synthetic LPAs and/or other simple lipid products may be used to modulate the activity of LIPH and/or other phospholipases may be used as potential small molecules to modulate hair growth. It will be understood that compounds useful in treatment compositions and methods of the invention may be chemically modified using standard methods to increase delivery, increase half-life, etc.

Treatment compositions and/or methods of the invention may also include, in some aspects, the application of compositions of phospholipases and PA together or alone for stimulation of hair growth. Because the function of LIPH is essential in hair bulge stem cells the manipulation by LIPH and homologous phospholipases may also potentially regulate stem cell development, cycling and differentiation of hair cells.

Some of the foregoing methods of the invention contemplate gene therapy. A procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell(s) of a subject which contains a defective copy of the gene, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art. In vivo gene therapy using vectors such as adenovirus, retroviruses, herpes virus, and targeted liposomes also is contemplated according to the invention.

In preferred embodiments, a virus vector for delivering a nucleic acid molecule encoding an LIPH polypeptide is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol. 26:1951-1959, 1996). In preferred embodiments, the virus vector is an adenovirus.

Another preferred virus for certain applications is the adeno-associated virus, a to double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimising the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired polypeptides, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991).

Preferably the foregoing nucleic acid delivery vectors: (1) contain exogenous genetic material that can be transcribed and translated in a mammalian cell and that can suppress an LIPH-associated disorder (e.g., a hair growth disorder), and preferably (2) contain on a surface a ligand that selectively binds to a receptor on the surface of a target cell, such as a mammalian cell, and thereby gains entry to the target cell.

Various techniques may be employed for introducing nucleic acid molecules of the invention into cells, depending on whether the nucleic acid molecules are introduced in vitro or in vivo in a host. Such techniques include transfection of nucleic acid molecule-CaPO₄ precipitates, transfection of nucleic acid molecules associated with DEAE, transfection or infection with the foregoing viruses including the nucleic acid molecule of interest, liposome-mediated transfection, and the like. For certain uses, it is preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane polypeptide on the target cell or a ligand for a receptor on the target cell can be bound to or incorporated within the nucleic acid molecule delivery vehicle. Especially preferred are monoclonal antibodies. Where liposomes are employed to deliver the nucleic acid molecules of the invention, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

In addition to delivery through the use of vectors, LIPH nucleic acids may be delivered to cells without vectors, e.g. as “naked” nucleic acid delivery using methods known to those of skill in the art.

Various forms of the LIPH polypeptide or nucleic acid, as described herein, can be administered and delivered to a mammalian cell (e.g., by virus or liposomes, or by any other suitable methods known in the art or later developed). The method of delivery can be modified to target certain cells, and in particular, cell surface receptor molecules or antigens present on neuronal cells. Methods of targeting cells to deliver nucleic acid constructs are known in the art. An LIPH polypeptide can also be delivered into cells by expressing a recombinant protein fused with peptide carrier molecules, examples of which, though not intended to be limiting, are tat or antennapedia. These delivery methods are known to those of skill in the art and are described in U.S. Pat. No. 6,080,724, and U.S. Pat. No. 5,783,662, the entire contents of which are hereby incorporated by reference.

In addition to the methods described herein for delivering exogenous LIPH, expression of endogenous normal or mutant LIPH can be induced (e.g., upregulated) by the administration of chemicals or other molecules that specifically increase the level of LIPH mRNA and/or polypeptide. Such induction and/or upregulation of endogenous LIPH may occur through methods that include, but not limited to: (a) activation of the LIPH promoter, (b) stabilization of LIPH mRNA, (c) increased translation of LIPH polypeptide and (d) stabilization of LIPH polypeptide.

The invention further provides efficient methods of identifying pharmacological agents or compounds for agents which mimic the functional activity of an LIPH molecule. Such LIPH functional activities include hair growth. Generally, the screening methods involve assaying for compounds which modulate (up-regulate) an LIPH functional activity.

The ability to screen drug compounds substances in vitro to determine the effect of the drug on specific LIPH function and/or hair growth should permit the development and screening of disorder-specific drugs. The invention also provides methods for identifying compounds that bind modulate hair growth. For example, LIPH polypeptides can be used in assays to determine the effect of a compound of LIPH activity and hair growth. Such an assay can accommodate the rapid screening of a large number of compounds to determine which compounds, if any, are capable of modulating LIPH activity. Subsequently, more detailed assays can be carried out with those compounds found to bind, to further determine whether such compounds act as modulators of hair growth.

Another application of the assays of the invention is the assay of test samples (e.g., hair follicle) for the presence or absence of a LIPH nucleic acid and/or polypeptide to assess the presence of one or more mutations in the molecules. Thus, for example, a hair follicle from a patient displaying symptoms related to a hair growth disorder can be assayed to determine if the observed symptoms are perhaps caused by a mutation in a LIPH molecule.

Assays contemplated by the present invention can be carried out in a variety of ways, as can readily be identified by those of skill in the art. In accordance with a further embodiment of the present invention, there is provided a bioassay for identifying compounds which modulate the activity of LIPH polypeptides of the invention. An example of such a bioassay includes: exposing cells containing DNA encoding LIPH polypeptides in a hair growth pathway, to one or more compounds whose ability to modulate the activity of the LIPH polypeptide is sought to be determined; and monitoring the cells for changes in LIPH activity and/or hair growth.

Assays of the invention can be used to identify compounds that modulate hair growth associate with LIPH polypeptides. According to this method, a LIPH polypeptide is contacted with an “unknown” or test compound, the activity of the LIPH polypeptide is monitored subsequent to the contact with the “unknown” or test substance, and those substances which increase or decrease the activity of the LIPH polypeptide is identified as a compound that modulates hair growth.

A wide variety of assays for pharmacological agents and compounds can be used in accordance with this aspect of the invention, including, labeled in vitro binding assays, electrophoretic mobility shift assays, immunoassays, cell-based assays such as two- or three-hybrid screens, expression assays, etc. The assay mixture comprises a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with polypeptides and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

In an exemplary assay, a mixture of the foregoing assay materials is incubated under conditions whereby, the presence of the presence of the candidate compound alters the expression or activity of the LIPH molecule or alters an aspect of the hair growth pathway that includes LIPH polypeptide. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of activity of a LIPH polypeptide and or the presence or absence of hair growth is detected by any convenient method available to the user. Detection may be effected in any convenient way for cell-based assays such as two- or three-hybrid screens.

It is contemplated that for high-throughput screening, each of the individual wells of samples contain the same cell type so that multiple compounds (obtained from different reagent sources in the apparatus or contained within different wells) can be screened and compared for modulating activity with respect to LIPH activity.

The invention further includes nucleic acid or protein microarrays with LIPH polypeptides or nucleic acids encoding such polypeptides. In this aspect of the invention, standard techniques of microarray technology are utilized to assess expression of the LIPH polypeptides and/or identify biological constituents that bind such polypeptides. Protein microarray technology, which is also known by other names including: protein chip technology and solid-phase protein array technology, is well known to those of ordinary skill in the art and is based on, but not limited to, obtaining an array of identified polypeptides or proteins on a fixed substrate, binding target molecules or biological constituents to the polypeptides, and evaluating such binding. See, e.g., G. MacBeath and S. L. Schreiber, “Printing Proteins as Microarrays for High-Throughput Function Determination,” Science 289(5485):1760-1763, 2000.

The invention will be more fully understood by reference to the following examples. These examples, however, are merely intended to illustrate the embodiments of the invention and are not to be construed to limit the scope of the invention.

### EXAMPLES

### Example 1

### Introduction

Positional cloning has been used to identify a mutant gene underlying a deficiency in human hair growth and scalp hair loss, the conditions termed hypotrichosis and alopecia. Males and females with the hair growth defect were selected via screening of ˜350,000 individuals in two populations. Homozygous mapping assigned the location of a mutant gene to a short genomic interval on chromosome 3q27. Mutation analysis identified the deletion in the Lipase H gene in this region caused by SINE-retroposon mediated recombination. Lipase H regulates the production of bioactive simple lipids and is expressed in hair follicles. The unexpected function found for the phospholipase implies a novel molecular mechanism controlling hair growth and development.

Unlike other genes implicated previously in human alopecia, this discovery 1) predict small molecules (simple lipids, essentially LPA isoforms) which may be easy to apply for hair treatment 2) identify the gene (and essential molecular pathway in hair follicle) specifically regulating the hair growth, but not other biological processes. Thus, the elaboration of this pathway is anticipated to have no or critically diminished side effects. The invention includes, in some aspects, the application of the small lipids, the products of lipases, for treatment and prevention of boldness and stimulation or vice versa inhibition of hair growth.

### Methods and Results

In a common population certain forms of inherited diseases may be missed because they lack informative large pedigrees and appropriate genealogical information. A direct large-scale epidemiologic screening has been performed in ethnic populations to reveal relatively frequent genetic disorders of unknown etiology. Previously, patients have been described patients (males and females) with hair growth deficiencies, the condition termed total hypotrichosis (8,9 OMIM604379). The comprehensive genetic epidemiological analysis of two ethnic groups of Caucasian origin in the Volga-Ural region (Mari El and Chuvash), was undertaken. The Mari population belongs to the Finno-Ugric group and Chuvash population to the Turks linguistic group. The ancestors of the Chuvash population were probably Volga Bulgars, extruded by Mongols from Volga Bulgaria, who settled in the territory occupied by Mari ancestral populations. During a genetic epidemiological study of 171,500 Mari individuals (total population size ˜324,000) and 178,722 Chuvash individuals (total population size ˜872,611) many individuals with a clinically similar form of hypotrichosis were identified.

Affected individuals are characterized from birth by deficiencies of hair growth (hypotrichosis) on the scalp and body. The growth of scalp hairs was retarded or arrested. The length of scalp hairs reached 2 cm in the temporal and frontal regions and a maximum of 5-6 cm in the occipital and parietal head regions). Hair loss on the scalp (alopecia) occasionally occurs in children and is slightly progressed with age, becoming common in the second and third decades of life. The scalp hairs were thin and the axilary and body hairs were sparse or absent. The histopathological analysis revealed abnormalities in the morphology of hair follicles and dystrophy and fragility of the hairs in analyzed individuals. This type of non-syndromic hair growth defect was not associated with any other pathologies. Biopsies of affected individuals showed slightly decreased numbers of large terminal follicles in comparison to normal individuals. No significant inflammatory infiltrate or scarring was seen in the affected subjects.

The parents of affected individuals were normal and the segregation frequency suggests autosomal-recessive type of inheritance. In total, fifty small nuclear families (fourteen from Mari and thirty six from Chuvash) were selected for further genetic analysis.

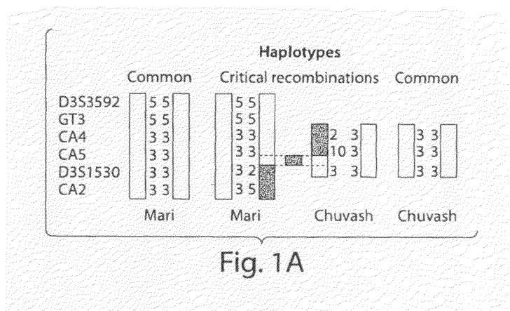

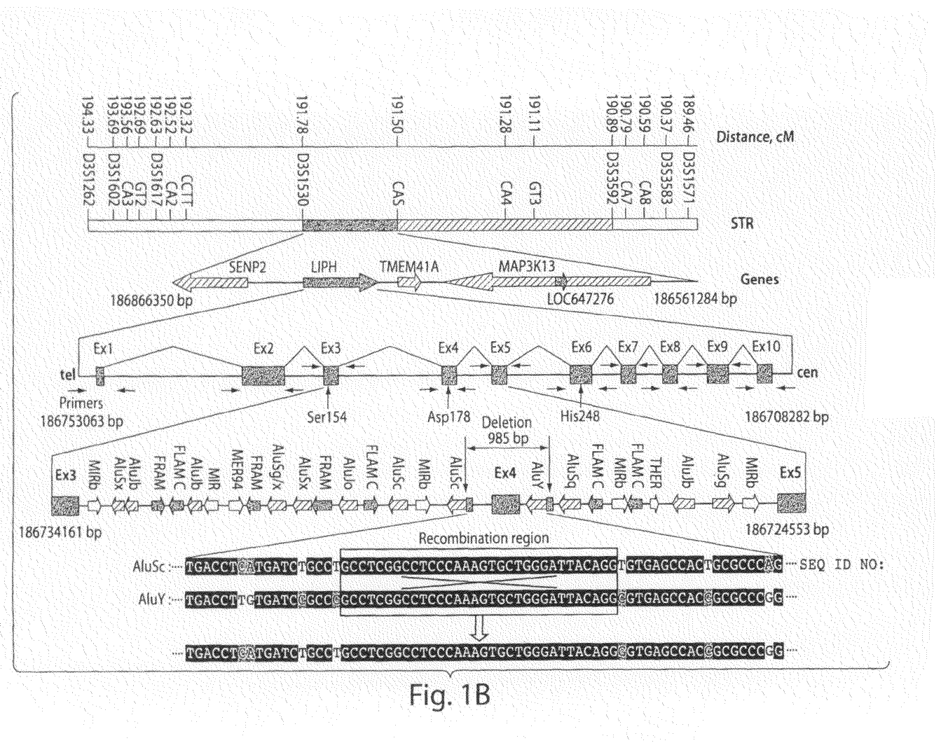

The primary genotyping was conducted in Mari families with a set of STR (simple tandem repeats) markers selected arbitrarily or from loci implicated in hair defects in human or rodents. The potential linkage to 3q27 was observed. Subsequent homozygous mapping in Mari families with known STR markers in this region suggested a relatively broad interval of ˜2.26 cM between D3S1617 and D3S3583. Four families with a suspected different form of hypotrichosis demonstrated no evidence for a linkage to this locus and were excluded from further analysis. The mapped interval contained 17 annotated genes. None of these genes have been previously implicated in function of hair follicles. The ETV5 and MAGEF1 genes located correspondingly at telomeric and centromeric boundaries of the mapped region and to expressed in skin were excluded by direct mutation analysis in affected individuals. In genealogical reconstructions we failed to reveal relationships between the families in the Mari and Chuvash populations. However, the finding of shared common STR haplotypes on 3q27 in unrelated affected individuals clearly indicates the inheritance of the mutation from a common ancient founder in both populations. Based on this assumption it appeared that examination of a high dense STR map on mutant chromosomes which are separated by many generations would critically reduce the genomic interval. Novel STRs were isolated and, in total, genotyping of 17 STR markers overlapping ˜5 cM between D3S1571 and D3S1262 was performed. The maximum two-point lod-scores were found for two closely located markers (CA)₅ (Zₘₐₓ=11.98, θ=0.00) and D3S1530 (Zₘₐₓ=9.09, θ=0.00). The analysis of shared and recombinant haplotypes in Mari families assigned the mutant gene to 0.89 cM interval flanked by D3S3592 and D3S1530 markers (FIGS. 1A, 1B). In combination with haplotypes in Chuvash families the gene was localized to short ˜0.28 cM genetic interval equating to only ˜305 kb. The mapped region contained four encoding genes (MAP3K13, TMEM41A, LIPH and SENP2) and one putative gene (LOC647276) with low presentation of SSTs in databases (FIG. 1B).

The entire encoding MAP3K13, TMEM41A and LIPH gene regions were sequenced including exon-intron junctions. No disease associated mutations were identified. We found, however, that in affected individuals the exon 4 of LIPH gene was not amplified with flanking intronic PCR primers. Clear PCR product for LIPH exon 4 was detected in the parents but not in any affected individuals. The data suggest the deletion of exon 4 persisting in heterozygous and heterozygous state (FIG. 1). To map the deletion breakpoints we designed a series of primers in the genomic region between exon 3 and exon 5. Using these primers, the PCR products of mutant alleles harboring deletion were generated and directly sequenced, the mapping of breakpoints determined the deletion of ˜985 bp that eliminates exon 4 and flanking intronic sequences. All families were examined and confirmed this deletion in the homozygous state in affected individuals and in heterozygous state in their parents. The genomic region between exon 3 and exon 5 is unusually densely saturated by SINE repeats. At least 26 SINE repeats were found within ˜10 kb interval that is almost 10 fold higher than the average density of SINES in the human genome (10). The sequencing of PCR product of the deletion allele and wild type predicted that the rearrangement is a result of unequal recombination between two copies of Alu-retroposons flanking exon 4. The recombination event occurred between highly homologous 5′-regions of the two members of distant ALU-subfamilies.

To determine the frequency of mutant allele 1118 chromosomes were tested in population sample collected irrespective of the hypotrichosis phenotype. As predicted, heterozygous carriers of LIPH deletion were detected in Chuvash region (n=I22 individuals, the mutant allele frequency p=0.033) and in Bashkirtastan (n=218 individuals, p°0.007) (other Volga-Ural region occupied by mixed population of ethnic Tatars, Bashkirs, Mari and Russians), but none in population group from distant geographic region (n=219 Russians, Moscow).

The predicted amino acid sequence of LIPH with deleted exon 4 remains in frame, however, the deletion eliminates evolutionary conserved domain. The LIPH (or mPA-PLA₁a) gene has a striking similarity to other members of the large triglyceride lipase gene family. Like all lipases, the LIPID contains the putative catalytic amino acid triad: Ser-154, Asp-178, and His-248 (11,12). The lipases, including LIPH, with artificial mutations replacing these residues have no enzymatic activity (12,13). Along with the Asp-178 several other amino acid-residues in the deleted LPH domain are completely conserved in all phospholipases. Thus, the deletion in homozygous state is anticipated to abolish the catalytic activity of LIPH.

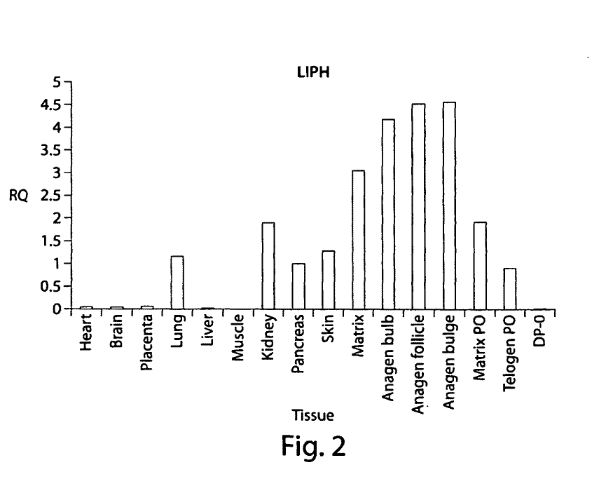

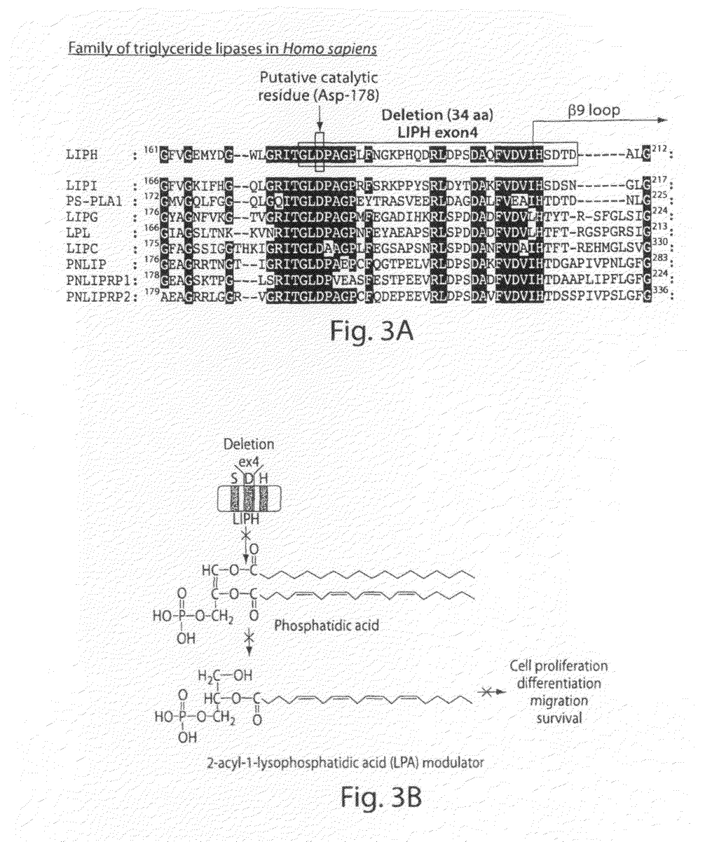

Since the role of lipase in hair growth is an unexpected finding, the question is raised as to whether the gene is expressed in hair follicle development. Real-time PCR and semi-quantitative RT-PCR for the LIPH gene were performed with RNA isolated from hair-follicles and for comparison with other tissues. Expression of LIPH was observed in the hair follicle bulge. The keratin was expressed in hair follicles and GADH housekeeping genes were used for controls. Elevated expression in hair follicle keratinocytes, but not in dermal papilla was found. The relative expression of LIPH in hair follicles was higher than in other tissues and was most prominent in stem cell-rich bulge region during anagen, the active stage of hair growth. The data further indicate the significance of LIPH in hair formation and growth. The LIPH expression was also clearly detectable in some other tissues (e.g., lung, pancreas and colon) (FIG. 2) (11,12). The LIPH forms subfamily of lipases with two homologous proteins LIPI and PS-PLA1. These three proteins share also common structure of short lids (12 amino acid residues) and only partial β9 domain. This structural feature may determine the phospholipase activity and deprive triglyceride lipase activity for this subfamily of proteins (14). The results indicate that the expression of the homologs of LIPH might compensate for LIPH loss of function in non-hair follicle somatic tissues in the affected individuals. Indeed, the PS-PLA1 homolog has significant expression in many human tissues but only weak or no expression in the hair follicle compartments [(15) and data not shown]. FIG. 3A shows a comparison of sequences of a family of triglyceride lipases in Homo sapiens. FIG. 3A shows conservation among family members and demonstrates that exon 4 encodes a highly conserved domain.

The one catalytic function suggested for the phospholipases is to generate the lysophosphatidic acids (LPA, 1- or 2-acyl-lysophosphatidic acids) from phospholipid (PL) (FIG. 3B) (16). The LPA was described as putative extracellular mediator of many biological functions, acting probably via EDG (endothelial cell differentiation gene) family of G-protein-coupled receptors (17). The potential role of LPA molecules was suggested as extracellular mediators of signaling in stimulation of cell proliferation, anti-apoptotic activities and cytoskeletal organization (12, 17-20). The LPA with saturated and unsaturated fatty acids may be produced in serum from platelet activation and in some other biological fluids and tissues by different biochemical mechanisms (16). The role of phospholipases and LPA in control of hair growth has not been previously demonstrated and the function of LIPH in vivo has not yet been elucidated. The LIPH expressed in insect Sf9 cells stimulates EDG7/LPA3 receptor for 2-acyl-LPA and participates in production of LPA rich in palmitoleic acid (16:1) and oleic acid (18:1) (12). It appears that the deletion of catalytic domain (loss-of function) of LIPH reduces or abolishes the production of LPA mediators in hair follicles and, thereby, affects the migration, differentiation or proliferation of keratinocytes culminating in arrest of hair growth. LPA-independent molecular mechanisms mediated by LIPH activity may also be factors in hair growth disorders. The identification of the genetic defect in phospholipase predicts a novel regulatory pathway in hair growth, re-cycling and putative therapeutic molecules to control hair loss or growth.

### REFERENCES FOR BACKGROUND, DETAILED DESCRIPTION, AND EXAMPLE 1

1. L. Alonso, E. Fuchs, Proc. Natl. Acad. Sci. U.S.A 100 Suppl 1, 11830 (2003).
2. K. A. Moore, I. R. Lemischka, Science 311, 1880 (2006).
3. S. Lyle et al., J. Cell Sef. 111 (Pt 21), 3179 (1995).
4. G. Cotsarelis, J. Clin. Invest 116, 19 (2006).
5. W. Ahmad et al., Science 279, 720 (1998).
6. A. Kljuic et al., Cell 113, 249 (2003).
7. E. Levy-Nissenbaum et al., Nat. Genet. 34, 151 (2003).
8. E. I. Rogaev, R. A. Zinchenko, G. Dvoryachikov, T. Sherbatich, E. K. Ginter, Lancet 354,1097 (1999).
9. OMIM database 604379 (htto-Hwww.nebi.nlm.nih.g_ov/enuez/dispomim.cgi?id˜604379)
10. E. S. Lander or al., Nature 409, 860 (2001).
11. W. Jin, U. C. Broedl, H. Monajemi, J. M. Glick, D. J. Rader, Genomics 80, 268 (2002).
12. H. Sonoda et al., J. Biol. Chem. 277, 34254 (2002).
13. M. Hiraoka, A. Abe, J. A. Shayman J:Lipid.Res, 46, 2441, 2005
14. T. Hiramatsu et al., J. Biol. Chem. 278, 49438 (2003)
15. J. Aoki et al. Biochmica et Biophisica Acta. 1582, 26 (2002)
16. J. Aoki, Semin. Cell Dev. Biol. 15, 477 (2004).
17. K. Bandoh et al., J Biol. Chem. 274, 27776 (1999).
18. D. R. Cerutis et al., Am, J. Physi 01273, L10 (1997).
19. G, A, Piazza, I L, Ritter, C. A. Baracka, Exp. Cell Res. 216, 51 (1995).
20. W. H. Moolenaar, Q. Kranenburg, F. R. Postma, G. C. Zondag, Curr. Opin. Cell Biol. 9, 168 (1997).

### Example 2

### Supporting Data for Example 1

### Methods

### Epidemiologic Analysis and Families

Genetic information was obtained about the affected individuals during a genetic epidemiological study performed in the Mari El and Chuvash populations (Russia). A detailed description of the methodology applied in the epidemiologic analysis was previously published (1). Briefly, the study of the distribution of hereditary diseases in each population involved three main steps. First, special registration cards that included a catalog of hereditary disorders (2) were distributed among local medical assistants, physicians and District Specialists (ophthalmologist, neurologist, dermatologist, etc). The data collected by the local medical personnel were combined with data collected from other medical sources about handicapped patients (e.g., specialized institutional schools for deaf and blind children). Second, all families were visited by clinical geneticists and those with a clearly nonhereditary pathology were excluded from the study. Finally, clinical evaluations were performed by neurologists, ophthalmologists, orthopedic surgeons, dermatologists, pediatricians and clinical geneticists. The collected data were subjected to segregation analysis.

In total, 350,222 individuals living in seven geographic areas in Mari and six geographic areas in the Chuvash Republics were screened. There was no significant difference in the frequency of the total hypotrichosis phenotype between the Mari and Chuvash populations (x2=1.25, p>0.05). An autosomal-recessive type of inheritance of this disorder was suggested (q=0.32+/−0.06, p=0.89). The frequency of the mutant gene was predicted to be at least 1.0-2.0% or higher in these populations. Fourteen families from Mari and 36 families from Chuvash were selected for molecular genetic analysis. No relationships were found between any of the pedigrees. In four families, the clinical presentations of hypotrichosis were distinct from the hypotrichosis phenotype observed in all other families and no evidence for linkage to chromosome 3827 was found in these families. STR genotyping of nuclear families with hypotrichosis was performed.

### Genotyping, Sequencing and Mutation Analysis.

Multiple STR (simple tandem repeats/microsatellites) markers across the human genome and from chromosome 3826-27 were selected from the Marshfield and Decode databases(//research.marshfieldclinic.org/genetics, www.decode.com/nrg1/markers/. In addition, new STRs were identified with the Tandem Repeats Finder (//tandem.bu.edu/trf/trf.submit.options.html) and tested for population polymorphisms. The PCR primers for these STRs are shown in Table 1.

TABLE 1
PCR primers for genotyping with 3q27 STR markers

| STR | Forward primer | SEQ ID NO> | Reverse primer | SEQ ID NO |
|---|---|---|---|---|
| D3S1571 | /6-FAM/ACAGTGGCTGATGCCTTT | 32 | CACAGGTGGGCACTACAT | 33 |
| D3S3583 | /6-FAM/TGCAAAGTCACAGATGTCCA | 34 | CGAGAGGCACCAGAGTGTT | 35 |
| CA8 | GCCTGGCCCAGAAAATAGAT | 36 | /6-FAM/TTCAAGGCCTACAACGTGATT | 37 |
| CA7 | /6-FAM/ACCAATGTTCAGGGGATGTC | 38 | GGTTGGGAGAGGAAGAGAGG | 39 |
| D3S3592 | /6-FAM/GCAGTTCTGAGTGATTTACCA | 40 | TCATCTGAGGTGTCTGATTG | 41 |
| GT3 | /6-FAM/TGGTGCAAAAGTGATTGTGG | 42 | GGGAGGATCTGTGGATTAGGA | 43 |
| CA4 | /6-FAM/AAGCCATGCCCATCACTTAG | 44 | TGGAAGGCAAGGCTGTATCT | 45 |
| CA5 | GCATTTTTGTTATCCTTGATTCC | 46 | /6-FAM/CCAATAGGCTTCAGGCAGAC | 47 |
| D3S1530 | /6-FAM/TTTAGCCTGGGTGACAGAGC | 48 | AACCGCATAAGCCAGTTGTT | 49 |
| GT1* | /6-FAM/TCTGGTGTCCAGATTTTGAAAG | 50 | CTTGGGTGCAGTTCTGGTCT | 51 |
| CCTT | /6-FAM/TGAGCAGGAAGGCAATAACA | 52 | CCTGGGTGACAGAGCAAGAC | 53 |
| CA2 | /6-FAM/AAGAAAGCCACCTCCCAAAC | 54 | CCCTTTGATTGCTTTCGAGT | 55 |
| D3S1617 | /6-FAM/CTGGTACAAAGAACAACAGTTTCC | 56 | TCTGTGAAAACAACATGGGC | 57 |
| GT2 | /6-FAM/TTAGGGTGACTTGCCCCTCT | 58 | CAACCCATGCCTTAAAGGTG | 59 |
| CA3 | TCATCTGCACCTCAGCCTTA | 60 | /6-FAM/GGCTGGGTAAAGGTTTTTGC | 61 |
| D3S1602 | AGAGCCTTCTATGGGTCTACAT | 62 | /6-FAM/AGCTCAACCTTCAAACATACATT | 63 |
| D3S1262 | /6-FAM/GGCCCTAGGATATTTTCAAT | 64 | AGTTTTTATGGACGGGGTTT | 65 |

*This STR marker, which has a low population heterozygosity, was not used for homozygous mapping of the critical genomic interval for the hypotrichosis locus.

PCR was performed in a total volume of 20 μl with 20-50 ng of genomic DNA and AmpliTaq Gold DNA polymerase (Applied Biosystems, Foster City, Calif.) under the following conditions: initial denaturation 94° C. 8 min, 5 cycles consisting of 94° C. 30 s, 58° C. 30 s, 72° C. 1 min, 30 cycles with 94° C. 30 s, 55° C. 30 s, 72° C. 1 min, final extension step on 72° C. 15 min. The STRs were tested on ABI371 or ABI3730 (Applied Biosystems, Foster City, Calif.) and examined on GeneMapper Software V4.0 (Applied Biosystems, Foster City, Calif.) by microsatellite analyses.

The sequencing of genes for mutational screening was performed using BigDye® Terminator v3.1 Cycle Sequencing Kit from Applied Biosystems, Foster City, Calif. To screen for mutations in candidate genes on 3827, intronic primer oligonucleotides were designed for each exon of C3orf65 (previous name LOC651498), MAGEFI, ETVS and MAP3K13, TMEM4IA, LIPH, SENP2 genes. The PCR amplification of LIPH using intronic primers flanking exon 4 failed in samples from affected individuals. Eight different pairs of PCR primers were designed for the LIPH genomic region to identify the boundaries of deletion of exon 4. Interspersed repeats and low complexity DNA sequences were identified by Repeat Masker web resource www.repeatmasker.org/cgi-bin/WEBRepeatMasker.

### Linkage Analysis

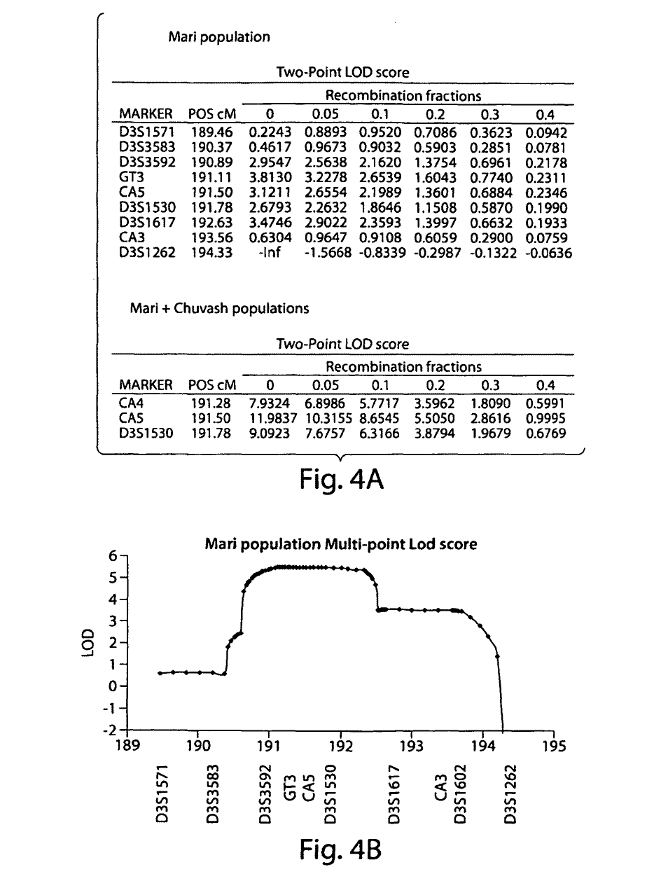

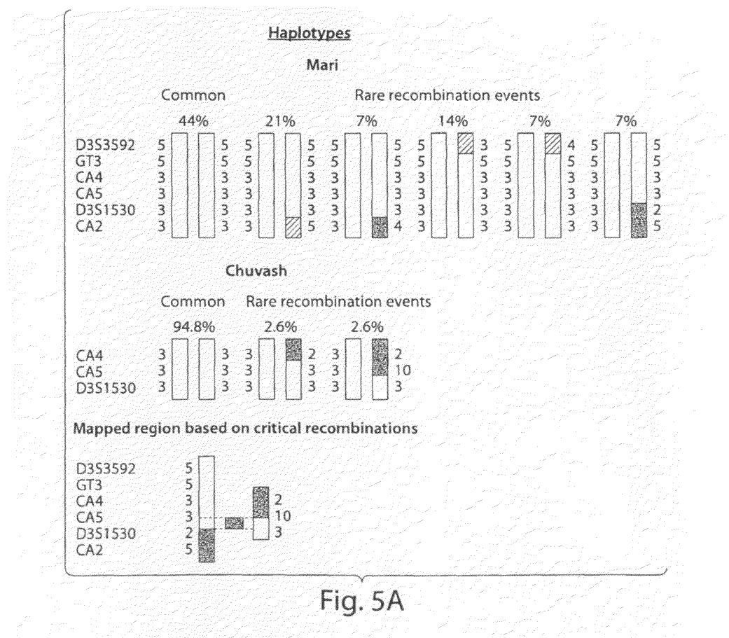

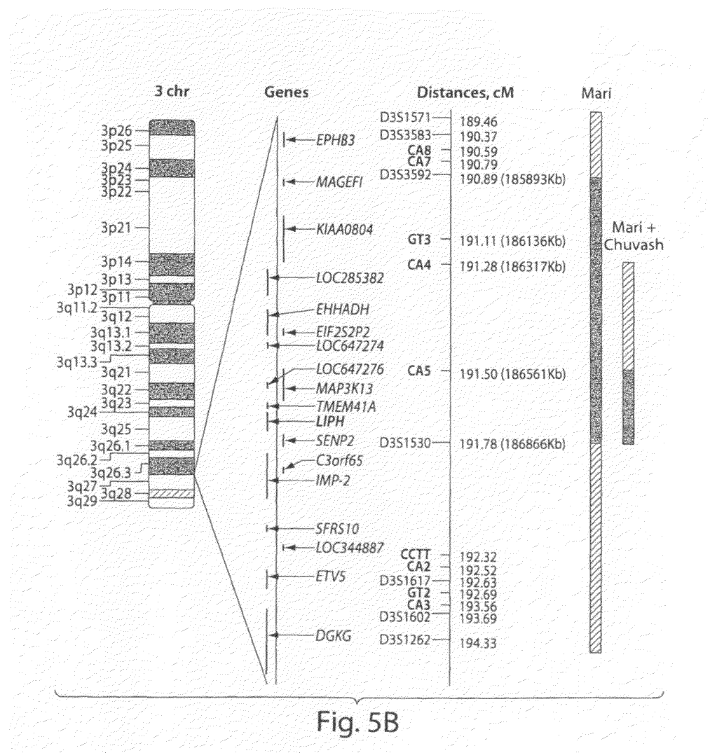

Two-point linkage analysis was calculated between each marker and the disorder using Superlink version 1.5 (//bioinfo.cs.technin.ac.il/superlink/ and Allegro Version 2.0 (3) (www.decode.com/software/allegro) and multi-point linkage analysis was performed using Allegro software. It was assumed that an equal frequency of STR alleles, autosomal-recessive inheritance of the disorder, full penetrance of the phenotype and a common mutation founder for all families analyzed. The frequencies for wild-type and mutant alleles were considered to be 0.99 and 0.01, respectively. Also, since de novo mutations of STRs could be misinterpreted as a recombination event, several closely located STRs in key putative recombination spots distal and proximal to the D3S1530-(CA)5 and D3S1530-D3S3592 intervals were tested. Results are shown in FIGS. 4A, 4B, 5A, and 5B.

### Identification of Deletion Breakpoints

To identify intronic boundaries for the LIPH deletion, the genomic sequence between exon 3 and exon 5 (9609 bp) was examined. The region contains multiple SINE-retrotransposons. To determine the deletion breakpoints, PCR was performed using a series of primers in the breakpoint region on DNA samples from parents and affected individuals. PCR with primers Del D and Del R was used for detection of the LIPH intragenic deletion. The PCR was used to identify the deletion in homozygous affected individuals and heterozygous carries. PCR with primers Del D [5′-CTGGCCTGACCAGTGAGTTT-3′ (SEQ ID NO:28)] and Del R [5′TTAGCCTGTCCTTCATTGAGC-3′ (SEQ ID NO:29)] yielded PCR products for mutant deletion allele and wild type allele. The PCR fragments corresponding to wild type and LIPH deletion alleles were separated by 1% agarose gel, purified via QIAquick Gel Extraction Kit (QIAGEN, Valencia, Calif.) and subjected to sequencing analysis. No PCR product was detected with the primers adjusted to exon 4 in affected homozygous individuals.

### RNA Preparation, Real-Time PCR and Semi-Quantitative PCR

The cDNA panel from human tissues was obtained from Clontech (Human MTCTM Panel I, Clontech laboratories, Mountain View, Calif.). Human hair follicles were obtained and dissected to retrieve the epithelial portions and dermal papilla as previously described (4, 5). Briefly, fresh samples of adult human scalp skin from plastic surgery procedures were obtained from the University of Massachusetts Cancer Center Tissue Bank. The skin (1×2 cm) was treated with 4 mg/ml Dispase (Sigma-Aldrich, St. Louis, Mo.) in DMEM (Invitrogen-Gibco, Carlsbad, Calif.) overnight at 4° C. Using forceps, hair follicles were plucked from the Dispase-treated skin, and segregated into telogen club hairs based on their morphology under a dissecting microscope. Plucked anagen follicles were dissected to remove the upper outer root sheath corresponding to the bulge region as well as the matrix area (bulb region). The keratinocytes from these regions were cultured on a feeder layer of 3T3 fibroblasts as previously described (4, 5). Dermal papilla cells were isolated from fresh untreated skin samples and cultured in Chang's media for up to two weeks (Irvine Scientific, Santa Ana, Calif.).

Total RNA was isolated from the tissue fragments and primary cell cultures using the RNeasy kit (QIAGEN, Valencia, Calif.), according to the manufacturer's instructions. The following specimens were used: skin—adult scalp skin, matrix—matrix area of plucked follicles, an. follicle—whole plucked follicle on anagene stage, an. bulge—dissected bulge region of anagen follicles which contains stem cells, bulb—bulb region of plucked follicles which contains matrix cells, telogen—plucked telogen follicles which contains stem cell region, DP—dermal papilla cells after 0, 1 or 12 days of culturing, the samples marked as PO are primary cultured keratinocytes from corresponding follicle areas.

First-strand DNA was synthesized using High-Capacity cDNA Archive Kit (Applied Biosystems, Foster City, Calif.) and RNase Inhibitor at a final concentration of 1.0 U/μl using 1 Ong/gl final RNA concentration in the volume of 50 gl. Reverse transcription was carried out under the standard conditions (25° C. 10 min, 37° C. 120 min). Semi-quantitative RT-PCR was performed with the following primers:

| KRT15 forward primer, | 5′-GGGTTTTGGTGGTGGCTTTG-3′; | (SEQ ID NO: 66) |
|---|---|---|
| KRT15 reverse primer, | 5′-TCGTGGTTCTTCTTCAGGTAGGC-3′; | (SEQ ID NO: 67) |
| GAPDH forward primer, | 5′-TGAAGGTCGGAGTCAACGGATTTGGT-3′; | (SEQ ID NO: 68) |
| GAPDH reverse primer, | 5′-CATGTGGGCCATGAGGTCCACCAC-3′ | (SEQ ID NO: 89) |
| (GAPDH gene, Human MTCTM Panel 1, Clontech Laboratories, Inc.); | | |
| LIPH forward primer, | 5′-CCATGGTCTTGAAGGAATTT-3′; | (SEQ ID NO 70) |
| LIPH reverse primer, | 5′-ACAGACCTCTGGTGGTCACA-3′; | (SEQ ID NO: 71) |
| LysoPLD forward primer, | 5′-GGACCCATGGAAGTTTGAAT-3′; | (SEQ ID NO: 72) |
| LysoPLD reverse primer, | 5′-GGAATCCGTAGGACATCTGC-3′; | (SEQ ID NO: 73) |
| LIPI forward primer, | 5′-GTGTGAGCTTAGGGGCTCAT-3′; | (SEQ ID NO: 74) |
| LIPI reverse primer, | 5′-CAGCCAGGTTGTTTATTTCCTC-3′; | (SEQ ID NO: 75) |
| PS-PLAI forward primer, | 5′-GACGCTGTCTGGATTGCTTT-3′; | (SEQ ID NO: 76) |
| PS-PLAI reverse primer, | 5′-TGGTTTATCTGGCATTGTGG-3′; | (SEQ ID NO: 77) |
| LPAI forward primer, | 5′-ATGGCACCCCTCTACAGTGA-3′; | (SEQ ID NO: 78) |
| LPAI reverse primer, | 5′-GCAGCAGAGGATCTGCCTAA-3′; | (SEQ ID NO: 79) |
| LPA2 forward primer, | 5′-TTGTCTTCCTGCTCATGGTG-3′; | (SEQ ID N0: 80) |
| LPA2 reverse primer, | 5′-CTCGGCAAGAGTACACAGCA-3′; | (SEQ ID NO: 81) |
| LPA3 forward primer, | 5′-CAACGTCTTGTCTCCGCATA-3′; | (SEQ ID NO: 82) |
| LPA3 reverse primer, | 5′-CACCTTTTCACATGCTGCAC-3′; | (SEQ ID NO: 83) |
| SIRPI forward primer, | 5′-CGAGAGCACTACGCAGTCAG-3′; | (SEQ ID NO: 84) |
| SIRPI reverse primer, | 5′-ACGTAGTCAGAGACCGAGCTG-3′; | (SEQ ID NO: 85) |
| SIRP2 forward primer, | 5′-GCCGGCCTAGCCAGTTCT-3′; | (SEQ ID NO: 86) |
| SIRP2 reverse primer, | 5′-AGGTCGTCTCCTGCGTTTC-3′; | (SEQ ID NO: 87) |
| SIRP3 forward primer, | 5′-CCAGCCCATCTGGCATTC-3′; | (SEQ ID NO: 88) |
| SIRP3 reverse primer, | 5′-AGCTCCAAAATCCACGAGAG-3′. | (SEQ ID NO: 89) |

PCR was performed in a final volume of 15 gl with 10-50 ng of cDNA, Taq DNA Polymerase (New England Biolabs, Ipswich, Mass.) under the following conditions: 94° C. 3 min, subsequent PCR cycles with 94° C. 30 s, 57° C. 30 s, 72° C. 40 s, final extension 72° C. 3 min. Since certain housekeeping genes may have different expression patterns in hair follicles than in other tissues, several housekeeping genes were uses for normalization (e.g., glyceraldehyde-3 phosphate dehydrogenase (GAPDH) and ubiquitin C (UBC)) in semi-quantitative RT-PCR and real-time PCR experiments.

The expression level of LIPH and PS-PLAT mRNA in the human tissue panel and hair follicle samples was examined by quantitative real-time PCR under the following conditions. PCR was carried out on an ABI Prism 7500 sequence detection system (Applied Biosystems, Foster City, Calif.) with TagMan Universal PCR Master Mix using TagMan Gene Expression Assays for LIPH (Hs00373748_ml) and UBC (Hs00824723_ml) in a total reaction volume of 25 μl. PCR cycling conditions were as follows: 95° C. for 10 min, 95° C. for 15 s and 60° C. for 1 min (40 cycles). The plate contained replicates for each tissue cDNA sample and a no-template water control. To evaluate the expression level of LIPH mRNA, the comparative threshold cycle (Ct) method was used. The relative amounts of LIPH mRNA were determined by normalizing to endogenous control gene (UBC) and relative to a calibrator sample (pancreas), and then calculating the 2^{-ΔΔct} for each sample.

### Histopathology

Punch biopsies of the scalp were obtained from three affected individuals. The biopsysamples were fixed in formalin, paraffin embedded and stained with hematoxylin and eosin. Multiple sections of each biopsy were examined under a light microscope by a dermatopathologist (S.L.).

cDNA Sequences And Gene Expression Constructs

To determine nucleotide sequence for complete encoding region of LIPH, RT PCR products for LIPH wild type (wt) and LIPH mutant type (with exon 4 deletion, AEx 4) were produced using pancreas RNA or RNA extracted from hair follicle bulge keratinocytes of affected individual. In addition, sequences for all exons of LIPH were determined using PCR products generated from genomic DNA.

A full length cDNA clone for wild type LIPH gene was obtained from Open Biosystems (Huntsville, Ala., USA) and was subcloned into the BamHI and XhoI sites of pcDNA6N5-His A and pcDNA4N5-His A (Invitrogen, Carlsbad, Calif.) using primers BamHI-d 5′-TTTTGGATCCTGTGAGCAAAATCCCACAGT-3′, (SEQ ID NO:90) XhoI-r 5′-TTTTCTCGAGCAACTGCAACTCTGGGCAAAG-3′ (SEQ ID NO:91). A cDNA clone of LIPH with deleted exon 4 (DEx 4) was produced as following:

1) Two PCR products were obtained using LIPH wt-pcDNA4N5-His A as a template and two pairs of primers:

(SEQ ID NO: 92)
BamHI-d, Aex4R1
5′-CTCCTTGTAGCCCAGTGCTGTAATTCTCCCCAGC-3
and
(SEQ ID NO: 93)
Aex4D2
5′-GCTGGGGAGAATTACAGCACTGGGCTACAAGGAG-3′,
XhoI-r.

2) Second round of PCR was performed on a mixture of gel-purified PCR products from the first round PCR using primers BamHI-d and XhoI-r.
3) The product of the second round PCR was subcloned into the PflMI-BspEI sites of LIPH wt-pcDNA4N5-His A, by substituting the wild type fragment with the AEx 4 fragment and resulting in LIPH-DEx 4 pcDNA4/V5-His A. The gene constructs were verified by DNA sequencing.

### Expression of Gene Constructs in Cultured Cells

Human keratinocyte stem cells isolated from the hair follicle bulge region were maintained in complete keratinocyte medium (KCM) at 37° C., 5% COz (S4, S5). Cell transfections with gene constructs were performed using Lipofectamine and Plus Reagent (Invitrogen, Carlsbad, Calif.) according to the manufacturer's protocol. 24 hours after transfection, cell lysates were prepared using modified Radioimmunoprecipitation (RIPA) Buffer, containing 50 mM Tris HCl, pH 7.4, 150 mM NaCl, 0.25% Sodium Deoxycholate, 1 mM EDTA, 1% NP-40, and complete Protease Inhibitor Cocktail (Roche Applied Science, Indianapolis, Ind.). Membranes were probed with primary monoclonal anti-V5 antibody (Invitrogen, Carlsbad, Calif.) and anti-mouse horse-radish-conjugated secondary antibody (Pierce Biotechnology, Rockford, Ill.) incubated with ECL-Plus Reagent (Amersham Biosciences, Buckinghamshire, UK) and exposed to X-Ray film.

### Results

### LIPH Mutation in Populations of the Volga-Ural Region.

The actual frequency of LIPH mutation can be estimated by direct genotyping of the population samples collected regardless of the pathology. The frequency of mutation in the Mari population was 0.030 (tested in 100 individuals) and in the Chuvash population was 0.033 (tested in 122 individuals). There are no religious or ethnic restrictions regarding marriages between different ethnic groups in the Chuvash and Mari El republics and based on the standard assumptions of the Hardy-Weinberg distribution of genotypes, one can expect the following numbers of heterozygous carriers and affected homozygous individuals (the demographics are according to the 1989 and 2002 Censuses) (www.perepis2002.ru/).

In the Chuvash republics (total population, 1,346,000; 889,268 ethnic Chuvash), the number of heterozygous mutant carriers should be 44,418 and 1,346 affected homozygous individuals (889 of them ethnic Chuvash). The total population of ethnic Chuvash (which includes compact groups living outside the Chuvash republic) is 906,922. Thus the number of all ethnic Chuvash heterozygous individuals should be 29,928 with 906 affected individuals.

In the Mari El republic (total population, 727,979; 312,178 ethnic Mari), the number of heterozygous mutant carriers should be 21,839 with 655 homozygous affected individuals (281 ethnic Mari). Calculations of the total ethnic Mari (643,700), including those living outside the Mari El republic, indicate that the number of heterozygous individuals should be 19,311 and homozygous affected individuals should be 579. In addition, the Chuvash and Mari republics were divided into several geographic regions and disease was found in all regions across the republics. Thus, the interpretation of the calculations of total Chuvash and Mari populations appears reasonable.

In summary, the calculations suggest that there are more than 50,000 heterozygous carriers and 1,400 homozygous individuals of Mari and Chuvash ethnicity and, perhaps, more than 2,000 affected individuals in total in this geographic region.

Additional analysis of ethnically unrelated 405 individuals of Russian ethnicity including Russians from Moscow (219 individuals) and the Southern (Rostov) and Central (Kostroma) provinces of Russia (186 individuals) was carried out. The mutation was not found in these Russian populations. Next, a Finno-Ugric group (174 individuals from Udmurt population) from the Volga-Ural region was tested and no mutation was found. Interestingly, however, when 345 individuals from a large population of Baskortostan (Volga-Ural region, 4,104,336 inhabitants including Turk-linguistic groups of Bashkirs and Tatars, Finno-Ugric Mari and Russians) were genotyped, heterozygous individuals and mutation were found with a frequency of 0.0004. Thus, the frequency of the mutation to accumulated in ethnic groups of the Volga-Ural region appears to be relatively high for an autosomal-recessive disorder. In this regard, the distribution of inherited diseases (6) and the history of Chuvash population might be of interest. The origin of Chuvash is believed to be related to the history of ancient Bulgars who moved to the Volga region. The Bulgar-Suvar groups were nomadic people and Chuvash may have a Hunnish heritage. The Chuvash language is a distant form of Turk and is the only surviving group of Altaic languages spoken by the Huns (7). The Asian origin of the Hun tribes and the invasion of Europe by Attila the Hun potentially might contribute to the flow of mutant genes that are found in Chuvash to other populations. Indeed, other rare autosomal-recessive disease, Chuvash polycythemia (MIM 263400), is caused by C598T (Arg200T) mutation in VHL gene (S8). The C598T mutation was found with frequency of 0.0167 among Chuvash and 0.0087 among Mari. Surprisingly the C598T mutation was found on the “Chuvash” like-haplotype chromosome also in several families of Asian and European ethnic origin and also accumulated on the Southern Italian Island of Ischia (9, 10) with no evidence of recent contact of Chuvash with other populations. It would be of interest to elucidate further the incidence of Mari-Chuvash hypotrichosis mutation in geographically distant ethnic populations.

### Mutation Identification

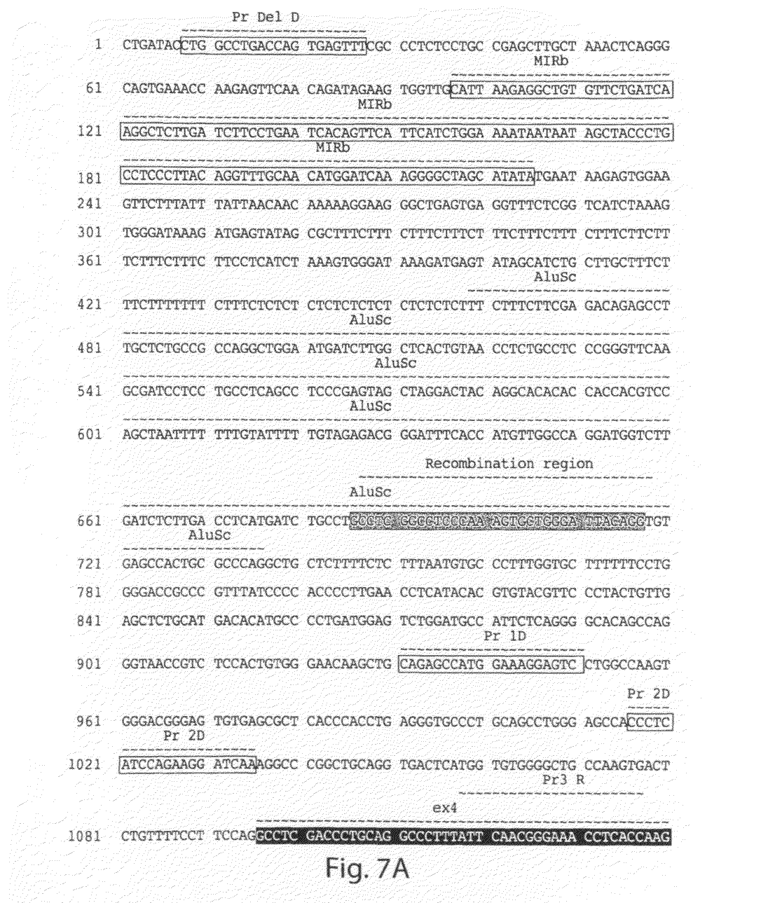

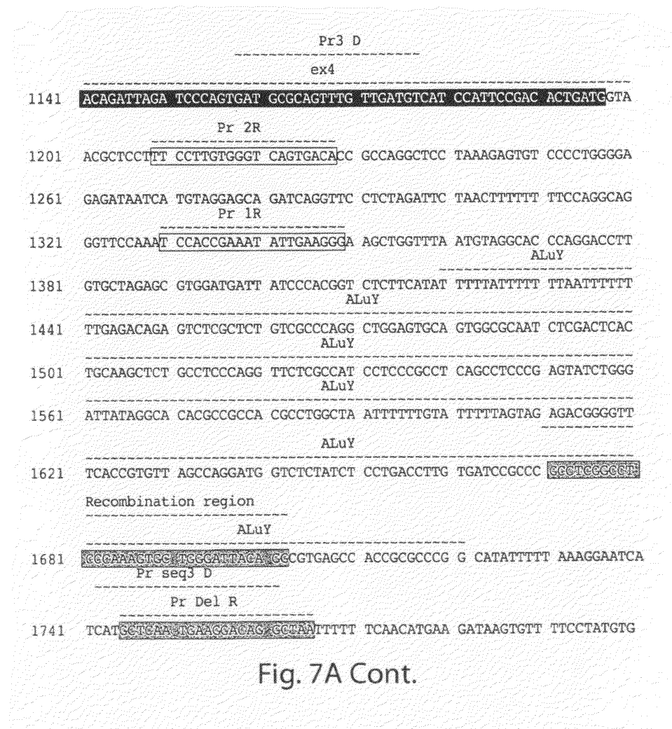

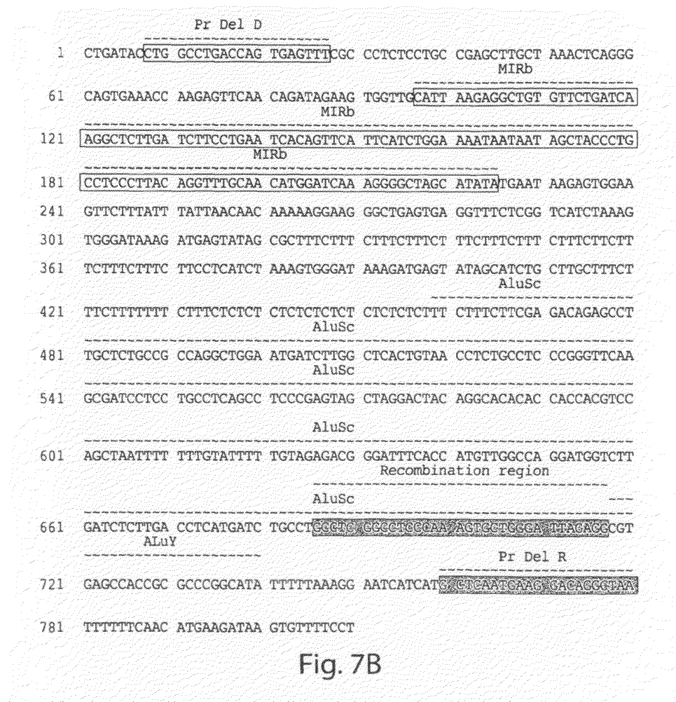

Assessment of the expression of mutant LIPH bearing a deletion of exon 4 was preformed. Results of RT-PCR analysis for mRNA extracted from keratinocytes of follicle bulge region from three individuals with hypotrichosis and a normal individual. Additional experiments were performed that included expression of cDNA3.1 constructs of wild-type and mutant LIPH (fused to V5-tag) in cultured hair follicle keratinocytes. Western blot results demonstrated efficient expression of LIPH protein (AEx 4) omitting 34 amino acid sequence of exon 4. The proteins were detected with primary monoclonal anti-V5 antibodies. The LIPH cDNA generated from mRNA of affected individual was sequenced and showed the deletion of exon 4 and fusion of exon 3 and exon 5 in mRNA transcript with no alteration of predicted open reading frame. These data confirm the results of genomic sequencing for the LIPH deletion allele (FIG. 7).

### Expression of Genes for Enzymes Regulating Synthesis of LPA

Several lines of evidence have indicated that membrane associated or secreted phospholipases may hydrolyze phospatidic acid (PA) associated with membranes (11-14). LPA can be produced from PA by LIPH (mPA-PLAIa) as well as by highly homologous LIPI (mPA-PLAID) and PS-PLAL. In addition, Lysophospholipase D (LysoPLD, identical to autotaxin) converts lysophospholipids to LPA and is the most important enzyme for LPA production in blood. Since these enzymes have overlapping functions and might be expected to compensate for the loss of one of them, the expression patterns for LIPH, PS-PL41 and LysoPLD in human somatic tissues and dissected regions from human hair follicles were compared. It was found that LysoPLD is highly expressed in many tissues tested, except the anagen hair follicle bulge. LIPI and PS-PL41 also have no detectable expression in follicle bulge, but are expressed in testis and other somatic tissues. Thus, among these phospholipases producing LPA only LIPH is sufficiently expressed in the hair follicle bulge region where hair follicle stem cells are located. These molecular data support the genetic findings and help explain why mutations in LIPH produce a hair-specific phenotype.

In order to determine if LPA receptors are also expressed in the hair follicle bulge, the levels of mRNA for six members of G-protein-coupled receptors (GPCR) have been analyzed, and it has been found that LPA1i LPA2 and LPA3 are expressed in this hair follicle compartment.

### REFERENCES FOR EXAMPLE 2

1. E. K. Ginter et al., Russian J. of Genetics (Genetika) 35, 385 (1999).
2. V. A. McKusick, Mendelian Inheritance in Man. A Catalog of Human Genes and Genetic Disorders, 12th edn. (Johns Hopkins Univ. Press, Baltimore, Md., 1998).
3. D. F. Gudbjartsson, T. Thorvaldsson, A. Kong, G. Gunnarsson, A. Ingolfsdottir, Nat. Genet. 37, 1015 (2005).
4. C. Roh, Q. Tao, S. Lyle, Physiol. Genomics 19, 207 (2004).
5. C. Roh, Q. Tao, C. Photopoulos, S. Lyle, J. Invest Dermatol. 125, 1099 (2005).
6. E. K. Ginter et al., Russian J. of Genetics (Genetika) 37, 692 (2001).
7. Encyclopaedia Britannica, http://www.britannica.com/.
8. S. O. Ang et al., Nat. Genet. 32, 614 (2002).
9. E. Liu et al., Blood 103, 1937 (2004).
10. S. Perrotta et al., Blood 107, 514 (2006).
11. T. Hiramatsu et al., J. Biol. Chem. 278, 49438 (2003).
12. H. Sonoda et al., J. Biol. Chem. 277, 34254 (2002).
13. F. Le Balle, M. F. Simon, S. Meijer, O. Fourcade, H. Chap, Adv. Enzyme Regul. 39, 275 (1999).
14. J. Aoki et al., J. Biol. Chem. 277, 48737 (2002).
15. F. Carriere et al., Biochim. Biophys. Acta 1376, 417 (1998).
16. W. Ahmad et al., Science 279, 720 (1998).
17. P. M. Clissold, C. P. Ponting, Trends Biochem. Sci. 26, 7 (2001).
18. X. Y. Wen et al., Hum. Mol. Genet. 12, 1131 (2003).

### Example 3

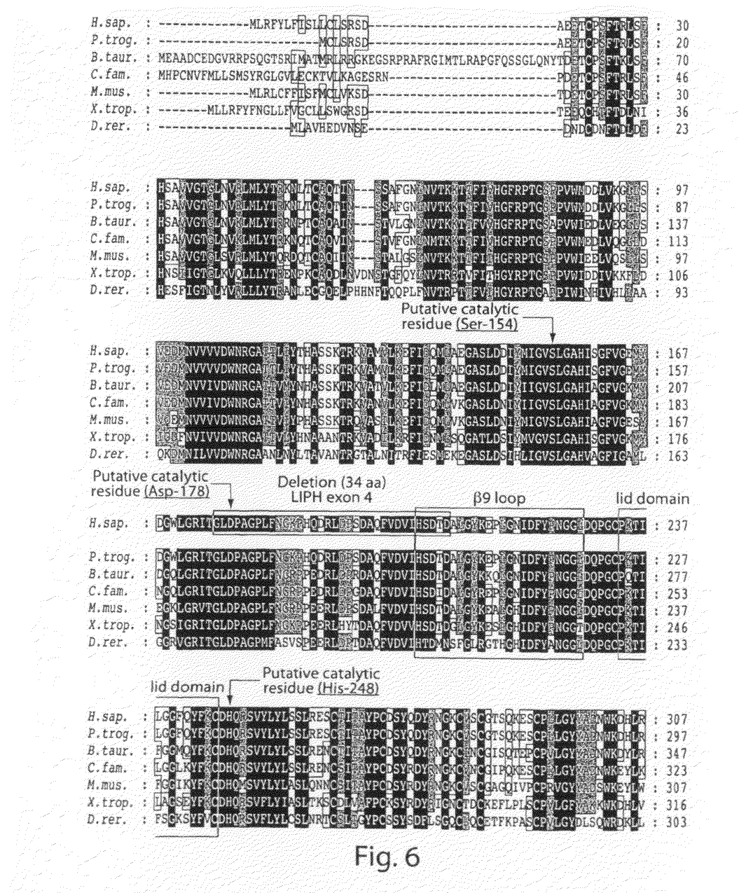

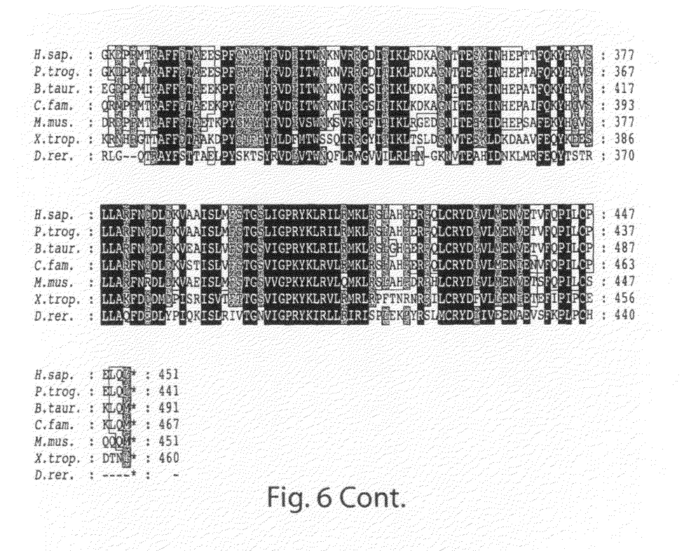

Multiple sequence alignments were performed. FIG. 6 shows multiple sequencing alignment of the human LIPH with orthologous members of LIPH family from the following vertebrate species: H.sap.—Homo sapiens NP_{—}640341 (SEQ ID NO:94); P.trog.—Pan troglodytes XP_{—}516924 (SEQ ID NO:95); B.taur.—Bos taurus XP_{—}589466 (SEQ ID NO:96); C.fam.—Canis familiaris XP_{—}545236 (SEQ ID NO:97); M.mus.—Mus musculus AAM18804 (SEQ ID NO:98); X.trop. Xenopus tropicalis NP_{—}001011098 (SEQ ID NO:99); D.rer.—Danio rerio XP_{—}687645 (SEQ ID NO:100). LIPH proteins have only partial (39 loop and a short Lid domain (indicated in boxes). These structural characteristics may determine the substrate selectivity of phospholipases [T. Hiramatsu et al., J. Biol. Chem. 278, 49438 (2003); F. Carriere et al., Biochim. Biophys. Acta 1376, 417 (1998)]. The evolutionarily conserved LIPH domain deleted in hypotrichosis patients contains catalytic Asp-178 residue and other evolutionary invariant amino acid residues. ClustalW program was used for multiple sequence alignment and GeneDoc program was used for shading. Some sequences showed amino acids of 100% identity, some showed 80% identity, and some showed 60% identify as can. The level of identify can be determined from FIG. 6. The hairless protein that is transcriptional factor regulating hair growth and inactivated in alopecia universalis [W. Ahmad et al., Science 279, 720 (1998)] contains JmJC domain frequently observed in DNA-binding proteins and identified in truncated form in Phospholipase A2P protein [P. M. Clissold, C. P. Ponting, Trends Biochem. Sci. 26, 7 (2001)]. However, no evidence was found for JmJC domain in the LIPH (Conserved Domain Database www.ncbi.nlm.nih.gov/Structure/cdd/Wrpsb.cgi NCBI).

Sequencing analysis was performed on the genomic region containing exon 4 and flanking introns of LIPH gene. FIG. 7A shows the sequence for wild-type allele (SEQ ID NO:30) and FIG. 7B shows the sequence of the mutant allele carrying the exon 4 deletion (SEQ ID NO:31). The sequences for PCR primers (Del D and Del R, SEQ ID NOs:28 and 29, respectively) used to detect the deletion and predicted regions of recombination between two ALU-elements are indicated on the wild type allele sequence.

Other aspects of the invention will be clear to the skilled artisan and need not be repeated here. Each reference cited herein is incorporated by reference in its entirety.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

Although the invention has been described in detail for the purpose of illustration, it is understood that such detail is solely for that purpose and variations can be made by those skilled in the art without departing from the spirit and scope of the invention which is defined by the following claims.

The contents of all references, patents and published patent applications cited throughout this application are incorporated herein by reference in their entirety.

We claim:

1. A method for diagnosing an LIPH-associated hair growth disorder in subject, the method comprising
assessing an LIPH gene or LIPH gene expression in a sample from the subject, wherein the presence of an alteration in the LIPH gene such that the gene encodes or expresses an LIPH polypeptide having a modified activity as compared to a wild-type LIPH polypeptide is diagnostic for an LIPH-associated hair growth disorder in the subject.

2.-3. (canceled)

4. The method of claim 1, wherein the LIPH gene expression is assessed by determining the level of LIPH mRNA or LIPH polypeptide in the cell.

5. The method of claim 1, wherein the alteration is in an exon of the LIPH gene sequence.

6. The method of claim 5, wherein the exon is exon 4 of the LIPH gene.

7. (canceled)

8. The method of claim 6, wherein the alternation of exon 4 is a deletion comprising all of the nucleotides of exon 4 of the LIPH gene.

9. The method of claim 6, wherein the alteration of exon 4 comprises the deletion or mutation of nucleotides that encode one or more of the Ser-154, Asp-178, and/or His-248 residues of the LIPH polypeptide.

10. The method of claim 6, wherein the alteration of exon 4 comprises the deletion or mutation of nucleotides that encode the Ser-154, Asp-178, and/or His-248 residues of the LIPH polypeptide.

11.-13. (canceled)

14. The method of claim 1, further comprising determining a treatment for the subject based, in part, on the diagnosis of the LIPH-associated hair growth disorder.

15. The method of claim 14, wherein the treatment comprises modifying a pathway comprising the LIPH polypeptide in a hair follicle cell of the subject an amount effective to modulate hair growth in the subject.

16. The method of claim 15, wherein modifying the pathway comprising the LIPH polypeptide comprises increasing a level of a simple lipid in the hair follicle cell.

17. The method of claim 16, wherein the simple lipid is a lysophosphatidic acid (LPA) or a modified LPA.

18. The method of claim 17, wherein the LPA is 1 or 2-acyl-lysophosphatidic acid.

19.-24. (canceled)

25. The method of claim 15, wherein modifying the pathway comprising LIPH polypeptide comprises increasing activity or expression of a homolog of LIPH.

26.-27. (canceled)

28. A kit for diagnosing a LIPH-associated hair growth disorder in a subject, the kit comprising a container containing a compound to assess an LIPH gene or LIPH gene expression in a sample from the subject, according to the method of claim 1.

29.-41. (canceled)

42. An isolated nucleic acid molecule that encodes (a) an LIPH polypeptide that possesses a lipase activity that is reduced from the level of lipase activity of a wild-type LIPH polypeptide, or (b) a full-length complement of thereof.

43. The isolated nucleic acid of claim 42, wherein the LIPH gene encodes an LIPH polypeptide having a deletion or substitution of all or part of the LIPH polypeptide sequence encoded by exon 4 of the LIPH gene.

44.-45. (canceled)

46. An expression vector comprising the isolated nucleic acid molecule of claim 42 operably linked to a promoter.

47. A host cell transformed or transfected with the expression vector of claim 46.

48. The host cell of claim 47, wherein the host cell is a hair follicle cell.

49. An isolated polypeptide encoded by the isolated nucleic acid molecule of claim 42.

50. (canceled)

51. The isolated polypeptide of claim 49, wherein the isolated polypeptide comprises an amino acid sequence encoded by the LIPH gene that is missing exon 4.

52. The method of claim 59, wherein the mutant LIPH gene encodes an LIPH polypeptide having a deletion or substitution of all or part of the LIPH polypeptide sequence.

53. The method of claim 52, wherein the deletion or substitution is in an exon of the LPH gene.

54. The method of claim 53, wherein the exon is exon 4 of the LIPH gene.

55.-58. (canceled)

59. A method for identifying a compound for increasing hair growth, the method comprising:
contacting the compound with a cell comprising a mutated LIPH gene, wherein the mutation reduces activity of LIPH polypeptide in the cell compared to a control cell with a non-mutated LIPH gene, and
determining the effect of the compound on LIPH polypeptide activity of the cell,
wherein a compound that increases LIPH polypeptide activity of the cell is identified as a compound that increases hair growth.


SEQUENCE LISTING (100 sequences)

SEQ ID NO: 1 - protein, 451 aa, Homo sapiens
MLRFYLFISLLCLSRSDAEETCPSFTRLSFHSAVVGTGLNVRLMLYTRKNLTCAQTINSSAFGNLNVTKKTTFIVHGFRPTGSPPVWMDDLVKGLLSVEDMNVVVVDWNRGATTLIYTHASSKTRKVAMVLKEFIDQMLAEGASLDDIYMIGVSLGAHISGFVGEMYDGWLGRITGLDPAGPLFNGKPHQDRLDPSDAQFVDVIHSDTDALGYKEPLGNIDFYPNGGLDQPGCPKTILGGFQYFKCDHQRSVYLYLSSLRESCTITAYPCDSYQDYRNGKCVSCGTSQKESCPLLGYYADNWKDHLRGKDPPMTKAFFDTAEESPFCMYHYFVDIITWNKNVRRGDITIKLRDKAGNTTESKINHEPTTFQKYHQVSLLARFNQDLDKVAAISLMFSTGSLIGPRYKLRILRMKLRSLAHPERPQLCRYDLVLMENVETVFQPILCPELQL

SEQ ID NO: 2 - DNA, 2115 bp, Homo sapiens
tcggcctcccaaagtgctgggattacagtggtcagccaccacacccagcttttcttgttgttgtttaatctttattttgtggttacaacatcataggagcttctctcctgtttttccagctgcttcgtagtgatagtgaggaacacccaaataacattgaccaagggtgagaaaccggtttaggcatttatagagcgggttaattctgcagggattgacacccacttggtttccctcttcaatacctagcctgatacctggcctgaccagtgagtttcgccctctcctgccgagcttgctaaactcagggcagtgaaaccaagagttcaacagatagaagtggttgcattaagaggctgtgttctgatcaaggctcttgatcttcctgaatcacagttcattcatctggaaaataataatagctaccctgcctcccttacaggtttgcaacatggatcaaatgggctagcatatatgaataagagtggaagttctttatttattaacaacaaaaaggaagggctgagtgaggtttctcggtcatctaaagtgggataaagatgagtatagcgctttctttctttctttctttctttctttctttctttctttctttctttcttctttctttctttcttcctcatctaaagtgggataaagatgagtatagcatctgcttgctttctttctttttttctttctctctctctctctctctctctttctttcttcgagacagagccttgctctgccgccaggctggaatgatcttggctcactgtaacctctgcctccctggttcaagcgatcctcctgcctcagcctcccgagtagctaggactacaggcacacaccaccacgtccagctaatttttttgtatttttgtagagacgggatttcaccatgttggccaggatggtcttgatctcttgacctcatgatctgcctgcctcggcctcccaaagtgctgggattacaggtgtgagccactgcgcccaggctgctcttttctctttaatgtgccctttggtgcttttttcctggggaccgcccgtttatccccaccccttgaacctcatacacgtgtacgttccctactgttgagctctgcatgacacatgcccctgatggagtctggatgccattctcaggggcacagccagggtaaccgtctccactgtgggaacaagctgcagagccatggaaaggagtcctggccaagtgggacgggagtgtgagcgctcacccacctgagggtgccctgcagcctgggagccaccctcatccagaaggatcaaaggcccggctgcaggtgactcatggtgtggggctgccaagtgactctgttttccttccaggcctcgaccctgcaggccctttattcaacgggaaacctcaccaagacagattagatcccagtgatgcgcagtttgttgatgtcatccattccgacactgatggtaacgctcctttccttgtgggtcagtgacaccgccaggctcctaaagagtgtcccctggggagagataatcatgtaggagcagatcaggttcctctagattctaacttttttttccaggcagggttccaaatccaccgaaatattgaagggaagctggtttaatgtaggcacccaggaccttgtgctagagcgtggatgattatcccacggtctcttcatatttttatttttttaattttttttgagacagagtctcgctctgtcgcccaggctggagtgcagtggcgcaatctcgactcactgcaagctcygcctcccaggttctcgccatcctcccgcctcagcctcccgagtagctgggattataggcacacgccgccacgcctggctaattttttgtatttttagtagagacggggtttcaccgtgttagccaggatggtctctatctcctgaccttgtgatccgcccgcctcggcctcccaaagtgctgggattacaggcgtgagccaccgcgcccggcatatttttaaaggaatcatcatgctcaatgaaggacaggctaattttttcaacatgaagataagtgttttcctatgtgcaactgggtttgcatgcatgatttcttagtgttgttggttctttttatttt

SEQ ID NO: 3 - protein, 417 aa, Homo sapiens
MLRFYLFISLLCLSRSDAEETCPSFTRLSFHSAVVGTGLNVRLMLYTRKNLTCAQTINSSAFGNLNVTKKTTFIVHGFRPTGSPPVWMDDLVKGLLSVEDMNVVVVDWNRGATTLIYTHASSKTRKVAMVLKEFIDQMLAEGASLDDIYMIGVSLGAHISGFVGEMYDGWLGRITALGYKEPLGNIDFYPNGGLDQPGCPKTILGGFQYFKCDHQRSVYLYLSSLRESCTITAYPCDSYQDYRNGKCVSCGTSQKESCPLLGYYADNWKDHLRGKDPPMTKAFFDTAEESPFCMYHYFVDIITWNKNVRRGDITIKLRDKAGNTTESKINHEPTTFQKYHQVSLLARFNQDLDKVAAISLMFSTGSLIGPRYKLRILRMKLRSLAHPERPQLCRYDLVLMENVETVFQPILCPELQL

SEQ ID NO: 4 - DNA, 984 bp, Homo sapiens
ggtgagaaaccggtttaggcatttatagagcgggttaattctgcagggattgacacccacttggtttccctcttcaatacctagcctgatacctggcctgaccagtgagtttcgccctctcctgccgagcttgctaaactcagggcagtgaaaccaagagttcaacagatagaagtggttgcattaagaggctgtgttctgatcaaggctcttgatcttcctgaatcacagttcattcatctggaaaataataatagctaccctgcctcccttacaggtttgcaacatggatcaaatgggctagcatatatgaataagagtggaagttctttatttattaacaacaaaaaggaagggctgagtgaggtttctcggtcatctaaagtgggataaagatgagtatagcgctttctttctttctttctttctttctttctttctttctttctttctttcttctttctttctttcttcctcatctaaagtgggataaagatgagtatagcatctgcttgctttctttctttttttctttctctctctctctctctctctctttctttcttcgagacagagccttgctctgccgccaggctggaatgatcttggctcactgtaacctctgcctcccgggttcaagcgatcctcctgcctcagcctcccgagtagctaggactacaggcacacaccaccacgtccagctaatttttttgtatttttgtagagacgggatttcaccatgttggccaggatggtcttgatctcttgacctcatgatctgcctgcctcggcctcccaaagtgctgggattacaggcgtgagccaccgcgcccggcatatttttaaaggaatcatcatgctcaatgaaggacaggctaattttttcaacatgaagataagtgttttcctatgtgcaactgggtttgcatgcatgatttcttagtgttgttggttctttttattttcttgagctgcaggtgttaa

SEQ ID NO: 5 - DNA, 99 bp, Homo sapiens
tttccatatgttgagattctacttattcatcagtttgttgtgcttgtcaagatcaggtaggttatttacaaactggttttcttaactgcttaaaaaata

SEQ ID NO: 6 - DNA, 432 bp, Homo sapiens
gtcgtgatgccatctaatttttattttcagacgcagaagaaacatgtccttcattcaccaggctgagctttcacagtgcagtggttggtacgggactaaatgtgaggctgatgctctacacaaggaaaaacctgacctgcgcacaaaccatcaactcctcagcttttgggaacttgaatgtgaccaagaaaaccaccttcattgtccatggattcaggccaacaggctcccctcctgtttggatggatgacttagtaaagggtttgctctctgttgaagacatgaacgtagttgttgttgattggaatcgaggagctacaactttaatatatacccatgcctctagtaagaccagaaaagtagccatggtcttgaaggaatttattgaccagatgttggtaagagaattctcttagatgattcagagctaag

SEQ ID NO: 7 - DNA, 201 bp, Homo sapiens
ttacaatgatggacctcttgaaattccaggcagaaggagcttctcttgatgacatttacatgatcggagtaagtctaggagcccacatatctgggtttgttggagagatgtacgatggatggctggggagaattacaggtaagtgcctgtgaatgggcatgggttggtagcctgggcagggtgttcactgggggagtatta

SEQ ID NO: 8 - DNA, 154 bp, Homo sapiens
agtataaaagcagaatgtgttctcttttagcactgggctacaaggagccattaggaaacatagacttctacccaaatggaggattggatcaacctggctgccccaaaacaatattgggaggtaagcctttatattttgtaaaatagtggtataa

SEQ ID NO: 9 - DNA, 256 bp, Homo sapiens
tgtgtatagaatatgtgatatagtattactaaatgcttcaaccgctctactctttcatcacttcttttacaaccaaggatttcagtattttaaatgtgaccaccagaggtctgtatacctgtacctgtcttccctgagagagagctgcaccatcactgcgtatccctgtgactcctaccaggattataggaatggcaagtgtgtcagctgcggcacgtcacaaaaagagtcctgtccccttctgggtaagtctaac

SEQ ID NO: 10 - DNA, 226 bp, Homo sapiens
caattttttaaacttgtttcataggctattatgctgataattggaaagaccatctaagggggaaagatcctccaatgacgaaggcattctttgacacagctgaggagagcccattctgcagtgagtggtgaatgtggttgttggcttttgcctaactctcattttggggatggctgagtctcagcttcctggctacttcttggcaaataagagccatgatgaagca

SEQ ID NO: 11 - DNA, 182 bp, Homo sapiens
ggggatcagttggtagtatctctctcttaccgtgttctctctctgttttgcagtgtatcattactttgtggatattataacatggaacaagaatgtaagaagaggggacattaccatcaaattgagagacaaagctggaaacaccacagaatccaaaatcaatcagtaagttgagttacaat

SEQ ID NO: 12 - DNA, 283 bp, Homo sapiens
gttaccagcttaccagtgacttgcaggcttcaaaactgctttctctcattagtagtctttatccatttttcagtattacttcttccttccagtgaacccaccacatttcagaaatatcaccaagtgagtctacttgcaagatttaatcaagatctggataaagtggctgcaatttccttgatgttctctacaggatctctaataggcccaaggtacaagctcaggattctccgaatgaagttaaggtcccttgcccatccggagaggtgagctgagggtacgg

SEQ ID NO: 13 - DNA, 224 bp, Homo sapiens
aattttgtatcttaaggatataatattatttactagaccattgcatttttataaattcatttacaaggatattttgtcttattttcccaggcctcagctgtgtcggtatgatcttgtcctgatggaaaacgttgaaacagtcttccaacctattctttgcccagagttgcagttgtaactgttgccaggacacatggccataaataatagaaagaaagctacaa

SEQ ID NO: 14 - DNA, 69 bp, Homo sapiens
tgacctcatgatctgcctgcctcggcctcccaaagtgctgggattacaggtgtgagccactgcgcccag

SEQ ID NO: 15 - DNA, 69 bp, Homo sapiens
tgaccttgtgatccgcccgcctcggcctcccaaagtgctgggattacaggcgtgagccaccgcgcccgg

SEQ ID NO: 16 - DNA, 69 bp, Homo sapiens
tgacctcatgatctgcctgcctcggcctcccaaagtgctgggattacaggcgtgagccaccgcgcccgg

SEQ ID NO: 17 - protein, 52 aa, Homo sapiens
GFVGEMYDGWLGRITGLDPAGPLFNGKPHQDRLDPSDAQFVDVIHSDTDALG

SEQ ID NO: 18 - protein, 52 aa, Homo sapiens
GFVGKIFHGQLGRITGLDPAGPRFSRKPPYSRLDYTDAKFVDVIHSDSNGLG

SEQ ID NO: 19 - protein, 52 aa, Homo sapiens
GMVGQLFGGQLGQITGLDPAGPEYTRASVEERLDAGDALFVEAIHTDTDNLG

SEQ ID NO: 20 - protein, 56 aa, Homo sapiens
GYAGNFVKGTVGRITGLDPAGPMFEGADIHKRLSPDDADFVDVLHTYTRSFGLSIG

SEQ ID NO: 21 - protein, 57 aa, Homo sapiens
GIAGSLTNKKVNRITGLDPAGPNFEYAEAPSRLSPDDADFVDVLHTFTRGSPGRSIG

SEQ ID NO: 22 - protein, 59 aa, Homo sapiens
GFAGSSIGGTHKIGRITGLDAAGPLFEGSAPSNRLSPDDANFVDAIHTFTREHMGLSVG

SEQ ID NO: 23 - protein, 58 aa, Homo sapiens
GEAGRRTNGTIGRITGLDPAEPCFQGTPELVRLDPSDAKFVDVIHTDGAPIVPNLGFG

SEQ ID NO: 24 - protein, 57 aa, Homo sapiens
GEAGSKTPGLSRITGLDPVEASFESTPEEVRLDPSDADFVDVIHTDAAPLIPFLGFG

SEQ ID NO: 25 - protein, 58 aa, Homo sapiens
AEAGRRLGGRVGRITGLDPAGPCFQDEPEEVRLDPSDAVFVDVIHTDSSPIVPSLGFG

SEQ ID NO: 26 - DNA, 2065 bp, Homo sapiens
tgagcaaaatcccacagtggaaactcttaagcctctgcgaagtaaatcattcttgtgaatgtgacacacgatctctccagtttccatatgttgagattctacttattcatcagtttgttgtgcttgtcaagatcagacgcagaagaaacatgtccttcattcaccaggctgagctttcacagtgcagtggttggtacgggactaaatgtgaggctgatgctctacacaaggaaaaacctgacctgcgcacaaaccatcaactcctcagcttttgggaacttgaatgtgaccaagaaaaccaccttcattgtccatggattcaggccaacaggctcccctcctgtttggatggatgacttagtaaagggtttgctctctgttgaagacatgaacgtagttgttgttgattggaatcgaggagctacaactttaatatatacccatgcctctagtaagaccagaaaagtagccatggtcttgaaggaatttattgaccagatgttggcagaaggagcttctcttgatgacatttacatgatcggagtaagtctaggagcccacatatctgggtttgttggagagatgtacgatggatggctggggagaattacagcactgggctacaaggagccattaggaaacatagacttctacccaaatggaggattggatcaacctggctgccccaaaacaatattgggaggatttcagtattttaaatgtgaccaccagaggtctgtatacctgtacctgtcttccctgagagagagctgcaccatcactgcgtatccctgtgactcctaccaggattataggaatggcaagtgtgtcagctgcggcacgtcacaaaaagagtcctgtccccttctgggctattatgctgataattggaaagaccatctaagggggaaagatcctccaatgacgaaggcattctttgacacagctgaggagagcccattctgcatgtatcattactttgtggatattataacatggaacaagaatgtaagaagaggggacattaccatcaaattgagagacaaagctggaaacaccacagaatccaaaatcaatcatgaacccaccacatttcagaaatatcaccaagtgagtctacttgcaagatttaatcaagatctggataaagtggctgcaatttccttgatgttctctacaggatctctaataggcccaaggtacaagctcaggattctccgaatgaagttaaggtcccttgcccatccggagaggcctcagctgtgtcggtatgatcttgtcctgatggaaaacgttgaaacagtcttccaacctattctttgcccagagttgcagttgtaactgttgccaggacacatggccataaataatagaaagaaagctacaaccacaggctgtttgaaagcttcacctcacctttctgcaaagcagaaaaagtatgaaaaaaaccaaggcttttttcagtagcgtcctatggatgtcacattgtacatcaaacaaccttgtgattataaaacgatcctgggaaggagcccctaactagggcaagtcagaaatagccaggctcgcagcagcgcagcgctgtgtctgctgtgtcctggggcctcccttgttccgacctgtcaattctgctgcctgtcacgcgggtggttctgcccatcgcggctgcgggtcaagcatcttcaagggaaggacggactggaggcctcaccgtggactcaactctgcattctccgtgccacattcctccagttcccacacgtagaagggaacgaaactgacgtctacctcatggggctgctgtgtgggtttgggaggcaaaaatctatgaagggttttttgaaatcccataggtgccacatctatgagatgtttgataaatgtgaatatgcttttacatttgggcttatctaatttgcaataagagagcctctctctatcaacaccagcttctctctcgggctgtttgctcagggaaggcaagaaagccacgtgctggccctctgccttctctaaagtgctgttggagcatggaggagctggaggagatggggatggactgaca

SEQ ID NO: 27 - DNA, 1506 bp, Homo sapiens
tgagcaaaatcccacagtggaaactcttaagcctctgcgaagtaaatcattcttgtgaatgtgacacacgatctctccagtttccatatgttgagattctacttattcatcagtttgttgtgcttgtcaagatcagacgcagaagaaacatgtccttcattcaccaggctgagctttcacagtgcagtggttggtacgggactaaatgtgaggctgatgctctacacaaggaaaaacctgacctgcgcacaaaccatcaactcctcagcttttgggaacttgaatgtgaccaagaaaaccaccttcattgtccatggattcaggccaacaggctcccctcctgtttggatggatgacttagtaaagggtttgctctctgttgaagacatgaacgtagttgttgttgattggaatcgaggagctacaactttaatatatacccatgcctctagtaagaccagaaaagtagccatggtcttgaaggaatttattgaccagatgttggcagaaggagcttctcttgatgacatttacatgatcggagtaagtctaggagcccacatatctgggtttgttggagagatgtacgatggatggctggggagaattacaggcctcgaccctgcaggccctttattcaacgggaaacctcaccaagacagattagatcccagtgatgcgcagtttgttgatgtcatccattccgacactgatgcactgggctacaaggagccattaggaaacatagacttctacccaaatggaggattggatcaacctggctgccccaaaacaatattgggaggatttcagtattttaaatgtgaccaccagaggtctgtatacctgtacctgtcttccctgagagagagctgcaccatcactgcgtatccctgtgactcctaccaggattataggaatggcaagtgtgtcagctgcggcacgtcacaaaaagagtcctgtccccttctgggctattatgctgataattggaaagaccatctaagggggaaagatcctccaatgacgaaggcattctttgacacagctgaggagagcccattctgcatgtatcattactttgtggatattataacatggaacaagaatgtaagaagaggggacattaccatcaaattgagagacaaagctggaaacaccacagaatccaaaatcaatcatgaacccaccacatttcagaaatatcaccaagtgagtctacttgcaagatttaatcaagatctggataaagtggctgcaatttccttgatgttctctacaggatctctaataggcccaaggtacaagctcaggattctccgaatgaagttaaggtcccttgcccatccggagaggcctcagctgtgtcggtatgatcttgtcctgatggaaaacgttgaaacagtcttccaacctattctttgcccagagttgcagttgtaactgttgccaggacacatggccataaataatagaaagaaagctacaaccacaggctgtttgaaa

SEQ ID NO: 28 - DNA, 20 bp, Artificial Sequence
  Synthetic Oligonucleotide
ctggcctgaccagtgagttt

SEQ ID NO: 29 - DNA, 21 bp, Artificial Sequence
  Synthetic Oligonucleotide
ttagcctgtccttcattgagc

SEQ ID NO: 30 - DNA, 1814 bp, Homo sapiens
ctgatacctggcctgaccagtgagtttcgccctctcctgccgagcttgctaaactcagggcagtgaaaccaagagttcaacagatagaagtggttgcattaagaggctgtgttctgatcaaggctcttgatcttcctgaatcacagttcattcatctggaaaataataatagctaccctgcctcccttacaggtttgcaacatggatcaaatgggctagcatatatgaataagagtggaagttctttatttattaacaacaaaaaggaagggctgagtgaggtttctcggtcatctaaagtgggataaagatgagtatagcgctttctttctttctttctttctttctttctttctttctttctttctttcttctttctttctttcttcctcatctaaagtgggataaagatgagtatagcatctgcttgctttctttctttttttctttctctctctctctctctctctctttctttcttcgagacagagccttgctctgccgccaggctggaatgatcttggctcactgtaacctctgcctccctggttcaagcgatcctcctgcctcagcctcccgagtagctaggactacaggcacacaccaccacgtccagctaatttttttgtatttttgtagagacgggatttcaccatgttggccaggatggtcttgatctcttgacctcatgatctgcctgcctcggcctcccaaagtgctgggattacaggtgtgagccactgcgcccaggctgctcttttctctttaatgtgccctttggtgcttttttcctggggaccgcccgtttatccccaccccttgaacctcatacacgtgtacgttccctactgttgagctctgcatgacacatgcccctgatggagtctggatgccattctcaggggcacagccagggtaaccgtctccactgtgggaacaagctgcagagccatggaaaggagtcctggccaagtgggacgggagtgtgagcgctcacccacctgagggtgccctgcagcctgggagccaccctcatccagaaggatcaaaggcccggctgcaggtgactcatggtgtggggctgccaagtgactctgttttccttccaggcctcgaccctgcaggccctttattcaacgggaaacctcaccaagacagattagatcccagtgatgcgcagtttgttgatgtcatccattccgacactgatggtaacgctcctttccttgtgggtcagtgacaccgccaggctcctaaagagtgtcccctggggagagataatcatgtaggagcagatcaggttcctctagattctaacttttttttccaggcagggttccaaatccaccgaaatattgaagggaagctggtttaatgtaggcacccaggaccttgtgctagagcgtggatgattatcccacggtctcttcatatttttatttttttaattttttttgagacagagtctcgctctgtcgcccaggctggagtgcagtggcgcaatctcgactcactgcaagctcygcctcccaggttctcgccatcctcccgcctcagcctcccgagtagctgggattataggcacacgccgccacgcctggctaattttttgtatttttagtagagacggggtttcaccgtgttagccaggatggtctctatctcctgaccttgtgatccgcccgcctcggcctcccaaagtgctgggattacaggcgtgagccaccgcgcccggcatatttttaaaggaatcatcatgctcaatgaaggacaggctaattttttcaacatgaagataagtgttttcctatgtg

SEQ ID NO: 31 - DNA, 810 bp, Homo sapiens
ctgatacctggcctgaccagtgagtttcgccctctcctgccgagcttgctaaactcagggcagtgaaaccaagagttcaacagatagaagtggttgcattaagaggctgtgttctgatcaaggctcttgatcttcctgaatcacagttcattcatctggaaaataataatagctaccctgcctcccttacaggtttgcaacatggatcaaaggggctagcatatatgaataagagtggaagttctttatttattaacaacaaaaaggaagggctgagtgaggtttctcggtcatctaaagtgggataaagatgagtatagcgctttctttctttctttctttctttctttctttcttctttctttctttcttcctcatctaaagtgggataaagatgagtatagcatctgcttgctttctttctttttttctttctctctctctctctctctctctctttctttcttcgagacagagccttgctctgccgccaggctggaatgatcttggctcactgtaacctctgcctcccgggttcaagcgatcctcctgcctcagcctcccgagtagctaggactacaggcacacaccaccacgtccagctaatttttttgtatttttgtagagacgggatttcaccatgttggccaggatggtcttgatctcttgacctcatgatctgcctgcctcggcctcccaaagtgctgggattacaggcgtgagccaccgcgcccggcatatttttaaaggaatcatcatgctcaatgaaggacaggctaattttttcaacatgaagataagtgttttcct

SEQ ID NO: 32 - DNA, 18 bp, Artificial Sequence
  Synthetic Oligonucleotide
acagtggctgatgccttt

SEQ ID NO: 33 - DNA, 18 bp, Artificial Sequence
  Synthetic Oligonucleotide
cacaggtgggcactacat

SEQ ID NO: 34 - DNA, 20 bp, Artificial Sequence
  Synthetic Oligonucleotide
tgcaaagtcacagatgtcca

SEQ ID NO: 35 - DNA, 19 bp, Artificial Sequence
  Synthetic Oligonucleotide
cgagaggcaccagagtgtt

SEQ ID NO: 36 - DNA, 20 bp, Artificial Sequence
  Synthetic Oligonucleotide
gcctggcccagaaaatagat

SEQ ID NO: 37 - DNA, 21 bp, Artificial Sequence
  Synthetic Oligonucleotide
ttcaaggcctacaacgtgatt

SEQ ID NO: 38 - DNA, 20 bp, Artificial Sequence
  Synthetic Oligonucleotide
accaatgttcaggggatgtc

SEQ ID NO: 39 - DNA, 20 bp, Artificial Sequence
  Synthetic Oligonucleotide
ggttgggagaggaagagagg

SEQ ID NO: 40 - DNA, 21 bp, Artificial Sequence
  Synthetic Oligonucleotide
gcagttctgagtgatttacca

SEQ ID NO: 41 - DNA, 20 bp, Artificial Sequence
  Synthetic Oligonucleotide
tcatctgaggtgtctgattg

SEQ ID NO: 42 - DNA, 20 bp, Artificial Sequence
  Synthetic Oligonucleotide
tggtgcaaaagtgattgtgg

SEQ ID NO: 43 - DNA, 21 bp, Artificial Sequence
  Synthetic Oligonucleotide
gggaggatctgtggattagga

SEQ ID NO: 44 - DNA, 20 bp, Artificial Sequence
  Synthetic Oligonucleotide
aagccatgcccatcacttag

SEQ ID NO: 45 - DNA, 20 bp, Artificial Sequence
  Synthetic Oligonucleotide
tggaaggcaaggctgtatct

SEQ ID NO: 46 - DNA, 23 bp, Artificial Sequence
  Synthetic Oligonucleotide
gcatttttgttatccttgattcc

SEQ ID NO: 47 - DNA, 20 bp, Artificial Sequence
  Synthetic Oligonucleotide
ccaataggcttcaggcagac

SEQ ID NO: 48 - DNA, 20 bp, Artificial Sequence
  Synthetic Oligonucleotide
tttagcctgggtgacagagc

SEQ ID NO: 49 - DNA, 20 bp, Artificial Sequence
  Synthetic Oligonucleotide
aaccgcataagccagttgtt

SEQ ID NO: 50 - DNA, 22 bp, Artificial Sequence
  Synthetic Oligonucleotide
tctggtgtccagattttgaaag

SEQ ID NO: 51 - DNA, 20 bp, Artificial Sequence
  Synthetic Oligonucleotide
cttgggtgcagttctggtct

SEQ ID NO: 52 - DNA, 20 bp, Artificial Sequence
  Synthetic Oligonucleotide
tgagcaggaaggcaataaca

SEQ ID NO: 53 - DNA, 20 bp, Artificial Sequence
  Synthetic Oligonucleotide
cctgggtgacagagcaagac

SEQ ID NO: 54 - DNA, 20 bp, Artificial Sequence
  Synthetic Oligonucleotide
aagaaagccacctcccaaac

SEQ ID NO: 55 - DNA, 20 bp, Artificial Sequence
  Synthetic Oligonucleotide
ccctttgattgctttcgagt

SEQ ID NO: 56 - DNA, 24 bp, Artificial Sequence
  Synthetic Oligonucleotide
ctggtacaaagaacaacagtttcc

SEQ ID NO: 57 - DNA, 20 bp, Artificial Sequence
  Synthetic Oligonucleotide
tctgtgaaaacaacatgggc

SEQ ID NO: 58 - DNA, 20 bp, Artificial Sequence
  Synthetic Oligonucleotide
ttagggtgacttgcccctct

SEQ ID NO: 59 - DNA, 20 bp, Artificial Sequence
  Synthetic Oligonucleotide
caacccatgccttaaaggtg

SEQ ID NO: 60 - DNA, 20 bp, Artificial Sequence
  Synthetic Oligonucleotide
tcatctgcacctcagcctta

SEQ ID NO: 61 - DNA, 20 bp, Artificial Sequence
  Synthetic Oligonucleotide
ggctgggtaaaggtttttgc

SEQ ID NO: 62 - DNA, 22 bp, Artificial Sequence
  Synthetic Oligonucleotide
agagccttctatgggtctacat

SEQ ID NO: 63 - DNA, 23 bp, Artificial Sequence
  Synthetic Oligonucleotide
agctcaaccttcaaacatacatt

SEQ ID NO: 64 - DNA, 20 bp, Artificial Sequence
  Synthetic Oligonucleotide
ggccctaggatattttcaat

SEQ ID NO: 65 - DNA, 20 bp, Artificial Sequence
  Synthetic Oligonucleotide
agtttttatggacggggttt

SEQ ID NO: 66 - DNA, 20 bp, Artificial Sequence
  Synthetic Oligonucleotide
gggttttggtggtggctttg

SEQ ID NO: 67 - DNA, 23 bp, Artificial Sequence
  Synthetic Oligonucleotide
tcgtggttcttcttcaggtaggc

SEQ ID NO: 68 - DNA, 26 bp, Artificial Sequence
  Synthetic Oligonucleotide
tgaaggtcggagtcaacggatttggt

SEQ ID NO: 69 - DNA, 24 bp, Artificial Sequence
  Synthetic Oligonucleotide
catgtgggccatgaggtccaccac

SEQ ID NO: 70 - DNA, 20 bp, Artificial Sequence
  Synthetic Oligonucleotide
ccatggtcttgaaggaattt

SEQ ID NO: 71 - DNA, 20 bp, Artificial Sequence
  Synthetic Oligonucleotide
ccagacctctggtggtcaca

SEQ ID NO: 72 - DNA, 20 bp, Artificial Sequence
  Synthetic Oligonucleotide
ggacccatggaagtttgaat

SEQ ID NO: 73 - DNA, 20 bp, Artificial Sequence
  Synthetic Oligonucleotide
ggaatccgtaggacatctgc

SEQ ID NO: 74 - DNA, 20 bp, Artificial Sequence
  Synthetic Oligonucleotide
gtgtgagcttaggggctcat

SEQ ID NO: 75 - DNA, 22 bp, Artificial Sequence
  Synthetic Oligonucleotide
cagccaggttgtttatttcctc

SEQ ID NO: 76 - DNA, 20 bp, Artificial Sequence
  Synthetic Oligonucleotide
gacgctgtctggattgcttt

SEQ ID NO: 77 - DNA, 20 bp, Artificial Sequence
  Synthetic Oligonucleotide
tggtttatctggcattgtgg

SEQ ID NO: 78 - DNA, 20 bp, Artificial Sequence
  Synthetic Oligonucleotide
atggcacccctctacagtga

SEQ ID NO: 79 - DNA, 20 bp, Artificial Sequence
  Synthetic Oligonucleotide
gcagcagaggatctgcctaa

SEQ ID NO: 80 - DNA, 20 bp, Artificial Sequence
  Synthetic Oligonucleotide
ttgtcttcctgctcatggtg

SEQ ID NO: 81 - DNA, 20 bp, Artificial Sequence
  Synthetic Oligonucleotide
ctcggcaagagtacacagca

SEQ ID NO: 82 - DNA, 20 bp, Artificial Sequence
  Synthetic Oligonucleotide
caacgtcttgtctccgcata

SEQ ID NO: 83 - DNA, 20 bp, Artificial Sequence
  Synthetic Oligonucleotide
caccttttcacatgctgcac

SEQ ID NO: 84 - DNA, 20 bp, Artificial Sequence
  Synthetic Oligonucleotide
cgagagcactacgcagtcag

SEQ ID NO: 85 - DNA, 21 bp, Artificial Sequence
  Synthetic Oligonucleotide
acgtagtcagagaccgagctg

SEQ ID NO: 86 - DNA, 18 bp, Artificial Sequence
  Synthetic Oligonucleotide
gccggcctagccagttct

SEQ ID NO: 87 - DNA, 19 bp, Artificial Sequence
  Synthetic Oligonucleotide
aggtcgtctcctgcgtttc

SEQ ID NO: 88 - DNA, 18 bp, Artificial Sequence
  Synthetic Oligonucleotide
ccagcccatctggcattc

SEQ ID NO: 89 - DNA, 20 bp, Artificial Sequence
  Synthetic Oligonucleotide
agctccaaaatccacgagag

SEQ ID NO: 90 - DNA, 30 bp, Artificial Sequence
  Synthetic Oligonucleotide
ttttggatcctgtgagcaaaatcccacagt

SEQ ID NO: 91 - DNA, 31 bp, Artificial Sequence
  Synthetic Oligonucleotide
ttttctcgagcaactgcaactctgggcaaag

SEQ ID NO: 92 - DNA, 34 bp, Artificial Sequence
  Synthetic Oligonucleotide
ctccttgtagcccagtgctgtaattctccccagc

SEQ ID NO: 93 - DNA, 34 bp, Artificial Sequence
  Synthetic Oligonucleotide
gctggggagaattacagcactgggctacaaggag

SEQ ID NO: 94 - protein, 451 aa, Homo sapiens; identical to SEQ ID NO: 1

SEQ ID NO: 95 - protein, 441 aa, Pan troglodytes
MCLSRSDAEETCPSFTRLSFHSAVVGTGLNVRLMLYTRKNLTCAQTINSSAFGNLNVTKKTTFIVHGFRPTGSPPVWMDDLVKGLLSVEDMNVVVVDWNRGATTLIYTHASSKTRKVAMVLKEFIDQMLAEGASLDDIYMIGVSLGAHISGFVGEMYDGWLGRITGLDPAGPLFNGKPHQDRLDPSDAQFVDVIHSDTDALGYKEPLGNIDFYPNGGLDQPGCPKTILGGFQYFKCDHQRSVYLYLSSLRESCTITAYPCDSYQDYRNGKCVSCGTSQKESCPLLGYYADNWKDHLRGKDPPMMKAFFDTAEESPFCMYHYFVDIITWNKNVRRGDITIKLRDKAGNTTESKINHEPTAFQKYHQVSLLARFNQDLDKVAAISLMFSTGSLIGPRYKLRILRMKLRSLAHPERPQLCRYDLVLMENVETVFQPILCPELQL

SEQ ID NO: 96 - protein, 491 aa, Bos Taurus
MEAADCEDGVRRPSQGTSRIMATMRLRRGKEGSRPRAFRGIMTLRAPGFQSSGLQNYTDETCPSFTKLSFHSAVVGTGLNVKLMLYTRRNPTCAQAINSTVLGNLNVTKKTTFVVHGFRPTGSAPVWIEDLVEGLLSVEDMNVVIVDWNRGATTVMYNHASSKTRKVATVLKEFIDLMLAEGASLDDIYIIGVSLGAHIAGFVGKMYDGQLGRITGLDPAGPLFNGRPPEDRLDPRDAQFVDVIHSDTDALGYKKQLGNIDFYPNGGLDQPGCPQTIFGGMQYFKCDHQRSVYLYLSSLRENCTITAYPCDSYQDYRNGKCINCGISQTEPCPVLGYYADNWKDYLREGDPPMIKAFFDTAEEKPFCIYHYFVDIITWNKNVRRGSITIKLKDKAGNTTESKINHEPATFQKYHQVSLLARFNQDLDKVEAISLVFSTGSVIGPRYKLRILRMKLRSLGHPERPQLCRYDLVLMENVETVFQPILCPKLQM

SEQ ID NO: 97 - protein, 467 aa, Canis familiares
MHPCNVFMLLSMSYRGLGVLECKTVLKAGESRNPDETCPSFTRLSFHSAVVGTGLNVKLMLYTRKNQTCAQVINSTVFGNLNMTKKTTFIVHGFRPTGSPPVWMEDLVQGLLDVEDMNVVIVDWNRGATTVIYNHASSKTRKVANVLKEFIDQMLVKGASLDNIYIIGVSLGAHIAGFVGKMYNGQLGRITGLDPAGPLFNGRPPEDRLDPGDAQFVDVIHSDIDALGYREPLGNIDFYPNGGLDQPGCPKTILGGLKYFKCDHQRSVYLYLSSLRENCSITAYPCDSYRDYRNGKCVNCGIPQKESCPLLGYYADNWKEYLKQRNPPMTKAFFDTAEEKPYCIYHYFVDIITWNKNIRRGSITIKLKDKAGNITESKINHEPAIFQKYHQVSLLARFNQDLDKVSTISLVFSTGSVIGPKYKLRVLRMKLRSLAHPERPQLCRYDLVLMENIENVFQPILCPKLQM

SEQ ID NO: 98 - protein, 451 aa, Mus musculus
MLRLCFFISFMCLVKSDTDETCPSFTRLSFHSAVVGTGLSVRLMLYTQRDQTCAQIINSTALGSLNVTKKTTFIIHGFRPTGSPPVWIEELVQSLISVQEMNVVVVDWNRGATTVIYPHASSKTRQVASILKEFIDQMLVKGASLDNIYMIGVSLGAHIAGFVGESYEGKLGRVTGLDPAGPLFNGRPPEERLDPSDALFVDVIHSDTDALGYKEALGHIDFYPNGGLDQPGCPKTIFGGIKYFKCDHQMSVYLYLASLQNNCSITAYPCDSYRDYRNGKCVSCGAGQIVPCPRVGYYADSWKEYLWDRDPPMTKAFFDTAETKPYCMYHYFVDIVSWNKSVRRGFITIKLRGEDGNITESKIDHEPSAFEKYHQVSLLARFNRDLDKVAEISLMFSTGSVVGPKYKLRVLQMKLRSLAHPDRPHLCRYDLVLMENVETSFQPILCSQQQM

SEQ ID NO: 99 - protein, 460 aa, Xenopus tropicales
MLLRFYFNGLLFVGCLLSWGRSDTEEQCHTFTDLNIHNSIIGTGLKVQLLLYTRENPKCAQDLNVDNSTGFQYLNVTRRTVFITHGYRPTGSPPVWIDDIVKKFLDIQDFNVIVVDWNRGATTVLYHNAAANTRKVADILKRFIDNMLSQGATLDSIYMVGVSLGAHISGFVGKMYNGSIGRITGLDPAGPLFNGKPPEERLHYTDAQFVDVVHSDTDGLGYKESLGHIDFYPNGGTDQPGCPKTILAGSEYFKCDHQRSVFLYIASLTKSCDLVAFPCKSYRDYRIGNCTDCKEFLPLSCPVLGFYADKWKDHLVKRNHPGTTAFFDTAAKDPYCIFHYYLDFMTWSSQIRRGYITIKLTSLDGNVTESKLDKDAAVFEQYKEESLLAKFDQDMDPISRISVTFTTGSVIGPKYKLRVLRMRLRPFTNRNRPILCRYDFVLLENIETEFIPIPCEDTNL

SEQ ID NO: 100 - protein, 440 aa, Danio rerio
MLAVHEDVNSEDNDCDNFTDLDFHESFIGTNLYVRLLLYTRANLECGQELPHHNFTQQPLFNVTRPTTFVIHGYRPTGAPPIWINHIVHLLAAQKDMNILVVDWNRGAANLNYLTAVANTRGTALNITRFIESMEKEGASLDSIHLIGVSLGAHVAGFIGAMLGGRVGRITGLDPAGPMFASVSPEERLDPTDAQFVDVLHTDMNSFGLRGTHGHIDFYANGGLDQPGCPKTIFSGKSYFVCDHQRSVFLYLCSLNRTCSLTGYPCSSYSDFLSGQCLQCETFKPASCPVLGYDLSQWRDKLLRLGQTRAYFSTTAELPYSKTSYRVDLVTWNQFLRWGVVILRLHNGKNVTEAHIDNKLMRFEQYTSTRLLAQFDEDLYPIQKISLRIVTGNVIGPRYKIRLLRIRISPLEKPYRSLMCRYDIIVEENAEVSFKPLPCH